(19) United States
(12) Patent Application Publication
(10) Pub. No.: US 2018/0291043 A1
(43) Pub. Date: Oct. 11, 2018

(54) BENZOCYCLOOCTENE-BASED AND INDENE-BASED ANTICANCER AGENTS

(71) Applicant: BAYLOR UNIVERSITY, Waco, TX (US)

(72) Inventors: Kevin G. PINNEY, Woodway, TX (US); Christine A. HERDMAN, Waco, TX (US); Rajendra P. TANPURE, Nasik Road (IN); Zhi CHEN, West Haven, CT (US)

(73) Assignee: BAYLOR UNIVERSITY, Waco, TX (US)

(21) Appl. No.: 16/003,565

(22) Filed: Jun. 8, 2018

Related U.S. Application Data
(62) Division of application No. 15/639,977, filed on Jun. 30, 2017.
(60) Provisional application No. 62/357,568, filed on Jul. 1, 2016.

(51) Int. Cl. C07F 9/06; A61K 31/661; C07F 9/6506; C07F 9/12; C07F 9/09; C07C 43/23

(52) U.S. Cl. CPC C07F 9/06; A61K 31/661; C07C 43/23; C07F 9/12; C07F 9/091; C07F 9/65062

(57) ABSTRACT

Benzocyclooctene (fused 6,8 ring system) analogues and corresponding indene (fused 6,5 ring system) analogues function as inhibitors of tubulin polymerization. The compounds are useful as anticancer agents in a new therapeutic approach for cancer treatment utilizing small-molecule inhibitors of tubulin polymerization that also act as vascular disrupting agents (VDAs).

This application is a divisional application of and claims priority to U.S. patent application Ser. No. 15/639,977, entitled “Benzocyclooctene-Based and Indene-Based Anticancer Agents,” filed on Jun. 30, 2017, which claims priority to U.S. Provisional Patent Application No. 62/357,568, entitled “Synthesis and Biological Evaluation of Benzocyclooctene-Based and Indene-Based Anticancer Agents that Function as Inhibitors of Tubulin Polymerization,” filed on Jul. 1, 2016, the entire contents of which are hereby incorporated by reference.

The present invention used in part funds from the National Cancer Institute of the National Institutes of Health, Grant No. 5R01CA140674. The United States Government has certain rights in the invention.

### BACKGROUND

The present disclosure relates to anticancer agents that function as inhibitors of tubulin polymerization.

The colchicine site located on the β-subunit of the α,β-tubulin heterodimer continues to serve as a rich and productive target for a wide-range of structurally diverse small-molecule anticancer agents. Through a direct binding interaction at the colchicine site, these compounds inhibit the polymerization (assembly) of the tubulin heterodimer into microtubules. Disruption of the inherent dynamic relationship between microtubules and tubulin heterodimers has led to two mechanistically distinct, yet complementary, cancer treatment strategies. One approach centers on the well-known cytotoxic effect that results when disruption of the tubulin-microtubule system, caused by treatment with small-molecule inhibitors of tubulin polymerization or depolymerization, impacts the ability of cancer cells to divide. A wide-range of small-molecule therapeutic agents has been discovered and developed that function as antiproliferative agents through this mechanism, including those shown in FIG. 1. As shown in FIG. 1, representative small-molecule anti-proliferative agents including colchicine, paclitaxel, CA4, vinblastine, OXi6196, KGP18, KGP156, OXi8006, and BNC105.

This approach, while productive as an anticancer strategy, has limitations and challenges since both neoplastic and healthy cells are potentially impacted. Targeting strategies such as the use of antibody-drug conjugates (ADCs) bearing active payloads have been successful with two FDA approved ADCs currently available. In another example, the targeting of tumor hypoxia with bioreductively activatable prodrug conjugates (BAPCs) has been explored with recent clinical trials involving PR-104 and TH-302 serving as representative examples. The structures of PR-104 and TH-302 are shown in FIG. 2.

A second mechanistic approach to cancer therapy involving inhibition of the tubulin-microtubule protein system centers on selective disruption of existing vasculature feeding tumors, leading to limitation of oxygen and nutrient delivery to the cells and impedance of the ability of these cells to clear cellular waste products. This ultimately leads to tumor necrosis. This approach to targeting existing tumor-feeding vasculature utilizes anticancer agents referred to as vascular disrupting agents (VDAs) and is unique and mechanistically distinct from the fairly well-established and more commonly described therapeutic approach employing angiogenesis inhibiting agents (AIAs) that impede the formation of new vessels. Rapid neovascularization occurs when tumors grow larger than about 1 mm³, as they can no longer acquire sufficient nutrients from the surrounding vasculature. Such tumors require their own vascular network to supply oxygen and nutrients and remove waste products. Because of their rapid growth and development, these vessels feature irregular branching and diameter, poor wall structure, abnormal bulges, and blind ends. This immature vasculature provides an attractive target for cancer therapy. Therapeutic agents that target tumor vasculature are referred to as vascular targeting agents (VTAs), which can be divided into the angiogenesis inhibiting agents (AIAs) and vascular disrupting agents (VDAs). AIAs inhibit the growth of new vasculature to the tumor, while VDAs damage already sprouted vessels. VDAs are sub-divided into biological agents and small-molecule therapeutics, and treatment with either of these entities leads to tumor necrosis. FIG. 3 shows several clinically relevant VDAs including CA4P, CA1P, BNC105, AVE8062, ZD6126, and Plinabulin (BeyondSpring Pharmaceuticals, New York N.Y.).

Colchicine, a natural product originally isolated from the autumn crocus, Colchicum autumnale, and the namesake for the colchicine site on tubulin, is a potent inhibitor of tubulin polymerization, but colchicine has a narrow therapeutic window limiting its development as an anticancer agent. Another natural product binding to the colchicine site on tubulin is CA4 (shown in FIG. 1). Originally isolated from the African bushwillow tree, Combretum caffrum, and synthesized by Pettit and co-workers, CA4 binds tightly to the colchicine site on tubulin and functions as a VDA. Combretastatin A-4P [(CA4P), FIG. 3] was synthesized as a water-soluble prodrug salt for therapeutic use.

### SUMMARY

The present disclosure pertains to anticancer agents that function as inhibitors of tubulin polymerization.

In particular, the present disclosure pertains to a variety of fused aryl-cycloalkyl and aryl-heterocyclic compounds that function as inhibitors of tubulin polymerization. The compounds are useful as anticancer agents in a new therapeutic approach for cancer treatment utilizing small-molecule inhibitors of tubulin polymerization that also act as vascular disrupting agents (VDAs).

Prominent among these compounds is a benzosuberene analogue (referred to herein as KPG18), which demonstrates potent cytotoxicity (sub-nM) against human cancer cell lines and functions (as its corresponding water-soluble prodrug salt) as a VDA in mouse models. Structure activity relationship considerations led to the evaluation of benzocyclooctyl [6,8 fused] and indene [6,5 fused] ring systems. Four benzocyclooctene and four indene analogues were prepared and evaluated biologically. Three of the benzocyclooctene analogues were active as inhibitors of tubulin polymerization (IC₅₀<5 μM), and benzocyclooctene phenol 23 was comparable to KGP18 in terms of potency. The analogous indene-based compound 31 also functioned as an inhibitor of tubulin polymerization (IC₅₀=11 μM) with reduced potency. The most potent inhibitor of tubulin polymerization from this group was benzocyclooctene analogue 23, and it was converted to its water-soluble prodrug salt 24 to assess its potential as a VDA. Preliminary in vivo studies, which utilized the MCF7-luc-GFP-mCherry breast tumor in a SCID mouse model, demonstrated that treatment with 24 (120 mg/kg) resulted in significant vascular shutdown, as evidenced by bioluminescence imaging (BLI) at 4 h post administration, and that the effect continued at both 24 and 48 h. Contemporaneous studies with CA4P, a clinically relevant VDA, were carried out as a positive control.

Accordingly, the present disclosure pertains to benzocyclooctene (fused 6,8 ring system) analogues and corresponding indene (fused 6,5 ring system) analogues.

### BRIEF DESCRIPTION OF DRAWINGS

FIG. 1 shows the structures of representative small-molecule anti-proliferative agents.

FIG. 2 shows the structures of PR-104 and TH-302.

FIG. 3 shows the structures of several clinically relevant VDAs.

FIG. 4 shows the structures of synthesized benzocylooctene analogues and indene analogues.

FIG. 5 shows a general synthetic scheme (Scheme 1) for the synthesis of benzocyclooctene analogues 5 and 6.

FIG. 6 shows a general synthetic scheme (Scheme 2) for synthesis of indanone analogues 11 and 12.

FIG. 7 shows a general synthetic scheme (Scheme 3) for synthesis of TBS-protected benzosuberone 14 and indanone 16.

FIG. 8 shows a general synthetic scheme (Scheme 4) for synthesis of target benzocyclooctene analogues 20, 21, and 23.

FIG. 9 shows a general synthetic scheme (Scheme 5) for conversion of analogue 23 to its corresponding phosphate prodrug salt 24.

FIG. 10 shows a general synthetic scheme (Scheme 6) for synthesis of target indene compounds 28, 29, and 31.

FIG. 11 shows a general synthetic scheme (Scheme 7) for synthesis of target indene water-soluble analogue 32.

FIG. 12 shows (A) graphs of light emission from individual MCF7-luc-GFP-mCherry breast tumors following administration of luciferin substrate, showing baseline, 4 h, 24 h and 48 h post administration of VDA analogue 24 (120 mg/kg) on the left and CA4P (120 mg/kg) on the right, and (B) photographs at selected time points for mice following administration of VDA analogue 24 (120 mg/kg) on the left and CA4P (120 mg/kg) on the right.

FIG. 13 shows histological assessment of tumor necrosis showing necrotic and viable regions at 48 h post treatment for (A) Analogue 24 (120 mg/kg), (B) CA4P (120 mg/kg), and (C) Saline.

### DETAILED DESCRIPTION OF PREFERRED EMBODIMENTS

The present disclosure relates to anticancer agents that inhibit tubulin polymerization.

Preferred embodiments include a benzocyclooctene analogue that functions as an inhibitor of tubulin polymerization and has a structure of:

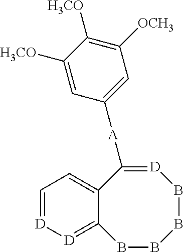

wherein A is a bond, such that the two ring structures are directly attached, or C═O, C═NH, or C═NR₁, where R₁ is any alkyl, aryl, or alkaryl substituent;
B is CH₂, NH, O, S, CH—OH, or CH—OR₂, wherein R₂ is CH₃, or PO₃⁻ to form a phosphate prodrug salt, or C—Z, wherein Z is halogen such as F, Cl, Br, or I; and
D is CH, N, C—OH, or C—OR₃, wherein R₃ is CH₃, any alkyl substituent, any alkoxy substituent, any alkyl or alkoxy substituent including heteroatoms, any carbonyl substituent, any ester substituent, or PO₃⁻ to form a phosphate prodrug salt, or C—Z, wherein Z is halogen such as F, Cl, Br, or I.

In preferred embodiments of the benzocylcooctene analogue above, each B and each D is independently the same or different. In these preferred embodiments, “alkyl” may refer to any alkyl group, including Me, Et, Pr, n-Pr, or i-Pr, “aryl” may refer to any aryl group, including phenyl, naphthyl, thienyl, indoyl, and the like, and “aralkyl” may refer to any alkyl group having at least one hydrogen atom replaced with an aryl group, including benzyl (CH₂Ph). “Me” may refer to methyl, “Et” may refer to ethyl, “Pr” may refer to propyl, “n-Pr” or “nPr” may refer to linear propyl, “i-Pr” may refer to isopropyl, “Ph” may refer to phenyl. The term “alkoxy” refers to an alkyl group attached to an oxygen atom. Examples of alkoxy include methoxy, ethoxy, propoxy, iso-propoxy, n-butoxy, tert-butoxy, neo-pentoxy and n-hexyloxy. The term “carbonyl substituent” refers to any ketone, aldehyde, or carboxylic acid substituent. An alkyl or alkoxy substitutent including heteroatoms is any alkyl or alkoxy substituent in which one of the carbon atoms is replaced with oxygen, nitrogen or sulfur.

Additional preferred embodiments of the present disclosure include an indene analogue that functions as an inhibitor of tubulin polymerization and has a structure of:

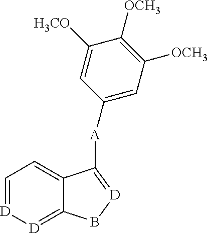

wherein A is a bond, such that the two ring structures are directly attached, or C═O, C═NH, or C═NR₁, where R₁ is any alkyl, aryl, or alkaryl substituent;
B is CH₂, NH, O, S, CH—OH, or CH—OR₂, wherein R₂ is CH₃ or PO₃⁻ to form a phosphate prodrug salt, or C—Z, wherein Z is halogen such as F, Cl, Br, or I; and
D is CH, N, C—OH, or C—OR₃, wherein R₃ is CH₃, any alkyl substituent, any alkoxy substituent, any alkyl or alkoxy substituent including heteroatoms, any carbonyl substituent, any ester substituent, or PO₃⁻ to form a phosphate prodrug salt, or C—Z, wherein Z is halogen such as F, Cl, Br, or I, and wherein at least one D is C—OH or C—OPO₃²⁻.

In preferred embodiments of the indene analogue above, each D is independently the same or different, but at least one D must be C—OH or C—OPO₃²⁻. In these preferred embodiments, “alkyl” may refer to any alkyl group, including Me, Et, Pr, n-Pr, or i-Pr, “aryl” may refer to any aryl group, including phenyl, naphthyl, thienyl, indoyl, and the like, and “aralkyl” may refer to any alkyl group having at least one hydrogen atom replaced with an aryl group, including benzyl (CH₂Ph). “Me” may refer to methyl, “Et” may refer to ethyl, “Pr” may refer to propyl, “n-Pr” or “nPr” may refer to linear propyl, “i-Pr” may refer to isopropyl, “Ph” may refer to phenyl. The term “alkoxy” refers to an alkyl group attached to an oxygen atom. Examples of alkoxy include methoxy, ethoxy, propoxy, iso-propoxy, n-butoxy, tert-butoxy, neo-pentoxy and n-hexyloxy. The term “carbonyl substituent” refers to any ketone, aldehyde, or carboxylic acid substituent. An alkyl or alkoxy substitutent including heteroatoms is any alkyl or alkoxy substituent in which one of the carbon atoms is replaced with oxygen, nitrogen or sulfur.

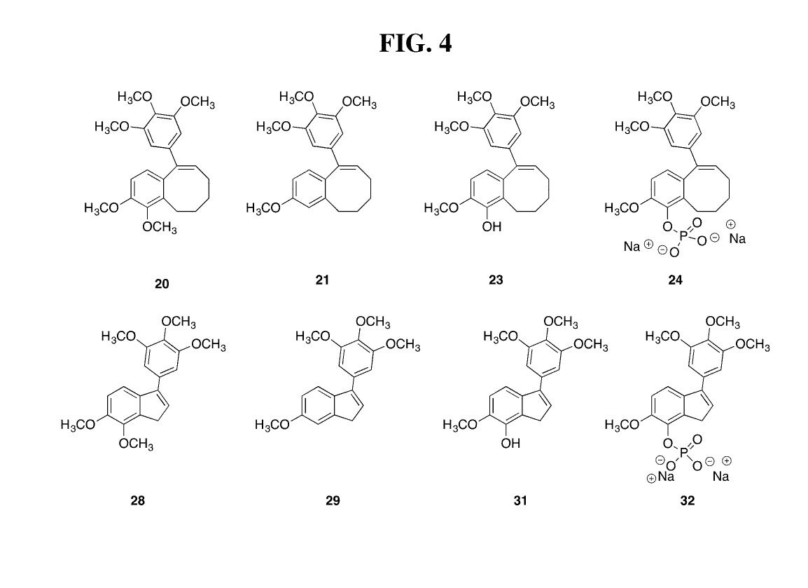

Preferred embodiments of the present disclosure include those anticancer agents, namely synthesized benzocylooctene analogues and indene analogues, shown in FIG. 4. Additional preferred embodiments pertain to methods for using the anticancer agents in a subject to inhibit tubulin polymerization and to treat cancer. The subject may be a cancer patient.

The exemplary compounds that function as inhibitors of tubulin polymerization described herein may occur in different geometric and enantiomeric forms, and both pure forms and mixtures of these separate isomers are included in the scope of this invention, as well as any physiologically functional or pharmacologically acceptable salt derivatives or prodrugs thereof. Production of these alternate forms would be well within the capabilities of one skilled in the art.

The current invention also pertains to methods of prevention or treatment of cancer, including the step of administering a compound that inhibits tubulin polymerization in accordance with preferred embodiments disclosed herein.

In another aspect of the present invention there is provided a pharmaceutical composition including a therapeutically effective amount of a compound, or its pharmaceutically acceptable salt or hydrate thereof, that inhibits tubulin polymerization as defined above and a pharmaceutically acceptable excipient, adjuvant, carrier, buffer or stabilizer. A “therapeutically effective amount” is to be understood as an amount of an exemplary inhibitor compound that is sufficient to show inhibitory effects on tubulin polymerization. The actual amount, rate and time-course of administration will depend on the nature and severity of the disease being treated. Prescription of treatment is within the responsibility of general practitioners and other medical doctors. The pharmaceutically acceptable excipient, adjuvant, carrier, buffer or stabiliser should be non-toxic and should not interfere with the efficacy of the active ingredient. The precise nature of the carrier or other material will depend on the route of administration, which may be oral, or by injection, such as cutaneous, subcutaneous, or intravenous injection, or by dry powder inhaler.

Pharmaceutical compositions for oral administration may be in tablet, capsule, powder or liquid form. A tablet may comprise a solid carrier or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. A capsule may comprise a solid carrier such as gelatin. For intravenous, cutaneous or subcutaneous injection, the active ingredient will be in the form of a parenterally acceptable aqueous solution which is pyrogen-free and has a suitable pH, isotonicity and stability. Those of relevant skill in the art are well able to prepare suitable solutions using, for example, isotonic vehicles such as sodium chloride solution, Ringer's solution, or lactated Ringer's solution. Preservatives, stabilizers, buffers, antioxidants and/or other additives may be included as required.

In another aspect, there is provided the use in the manufacture of a medicament of a therapeutically effective amount of tubulin polymerization inhibitor compound as defined above for administration to a subject.

The term “pharmacologically acceptable salt” used throughout the specification is to be taken as meaning any acid or base derived salt formed from hydrochloric, sulfuric, phosphoric, acetic, citric, oxalic, malonic, salicylic, malic, fumaric, succinic, ascorbic, maleic, methanesulfonic, isoethonic acids and the like, and potassium carbonate, sodium or potassium hydroxide, ammonia, triethylamine, triethanolamine and the like.

The term “prodrug” means a pharmacological substance that is administered in an inactive, or significantly less active, form. Once administered, the prodrug is metabolised in vivo into an active metabolite. In preferred embodiments, a prodrug is a water soluble phosphate salt.

The term “therapeutically effective amount” means a nontoxic but sufficient amount of the drug to provide the desired therapeutic effect. The amount that is “effective” will vary from subject to subject, depending on the age and general condition of the individual, the particular concentration and composition being administered, and the like. Thus, it is not always possible to specify an exact effective amount. However, an appropriate effective amount in any individual case may be determined by one of ordinary skill in the art using routine experimentation. Furthermore, the effective amount is the concentration that is within a range sufficient to permit ready application of the formulation so as to deliver an amount of the drug that is within a therapeutically effective range.

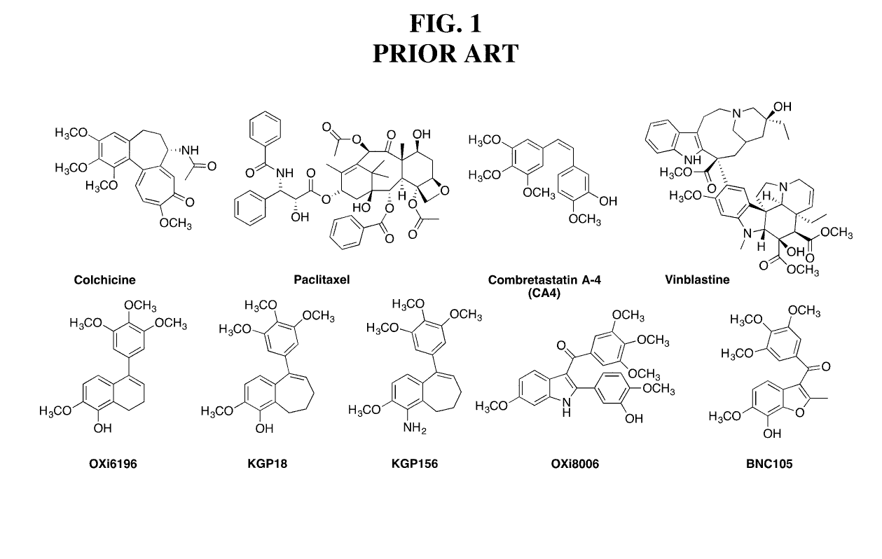

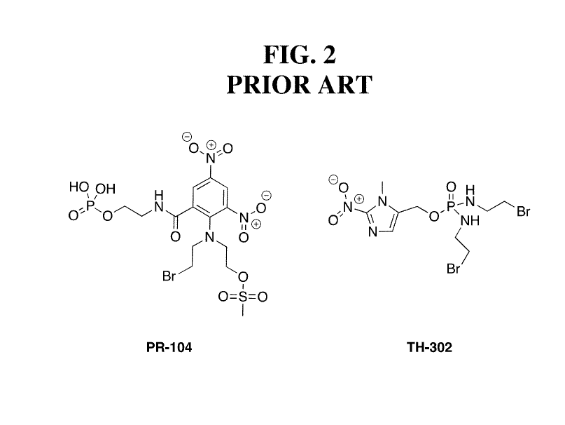

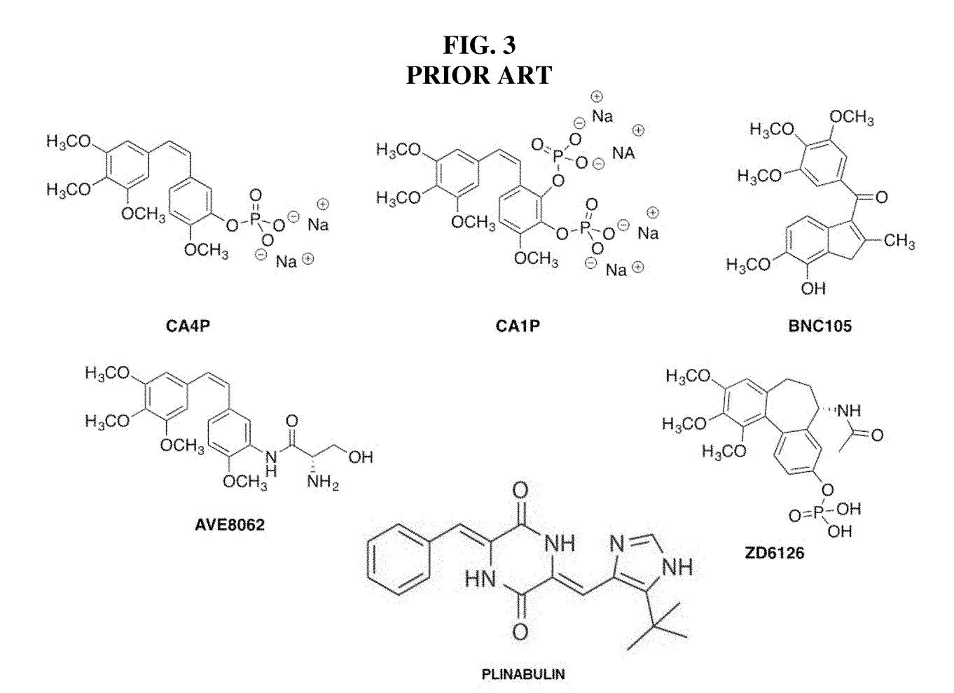

The current invention also pertains to methods of prevention or treatment of cancer, including the step of administering a compound that inhibits tubulin polymerization in accordance with preferred embodiments disclosed herein, in conjunction with an additional, secondary, supplemental, or combination therapy that is also intended for the prevention or treatment of cancer. These additional therapies include administration of at least one additional compound or pharmaceutical composition having anti-cancer activity, simultaneously with the administration of the present compounds. The additional compound can be any recognized compound, pharmaceutical composition, or drug that has demonstrated usefulness in the prevention or treatment of cancer, including but not limited to other compounds identified in FIG. 1-3. The additional therapies may also include radiation therapy or immunotherapy.

The following examples are included to demonstrate preferred embodiments of the invention. It should be appreciated by those of skill in the art that the techniques disclosed in the examples that follow present techniques discovered by the inventor to function well in the practice of the invention, and thus can be considered to constitute preferred modes for its practice. However, those of skill in the art should, in light of the present disclosure, appreciate that many changes can be made in the specific embodiments that are disclosed and still obtain a like or similar result without departing from the spirit and scope of the invention.

### Examples

### General Synthesis

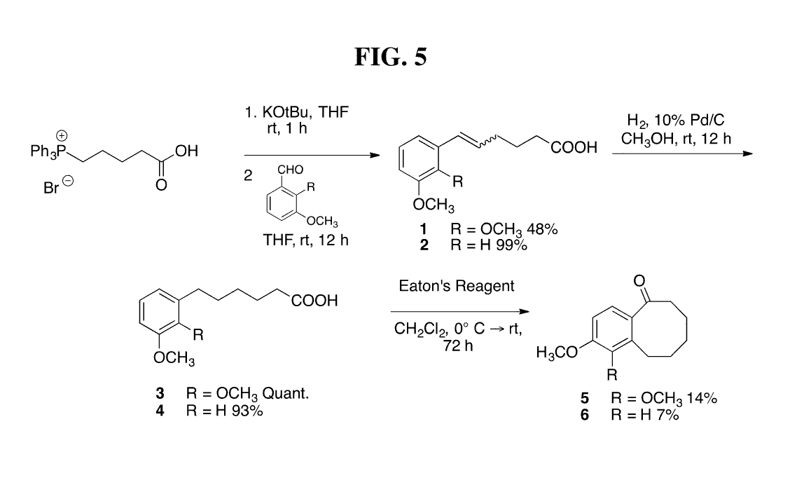

Four benzocyclooctene analogues and four indene analogues were synthesized. These analogues were prepared utilizing a synthetic strategy reminiscent of that employed for a variety of benzosuberene analogues (Tanpure et al., 2012; Herdman et al., 2015). The synthesis of each benzocyclooctene analogue was initiated with a Wittig olefination followed by catalytic hydrogenation to afford carboxylic acids 3 and 4, as shown in Scheme 1 in FIG. 5. A Friedel-Crafts intramolecular annulation was accomplished using Eaton's reagent (7.7 weight percent P₂O₅ in CH₃SO₃H) (Eaton et al., 1973), and the reaction was diluted and cooled in order to facilitate the construction of benzocyclooctones 5 and 6. Without dilution and cooling, the desired product was not formed and only starting material remained.

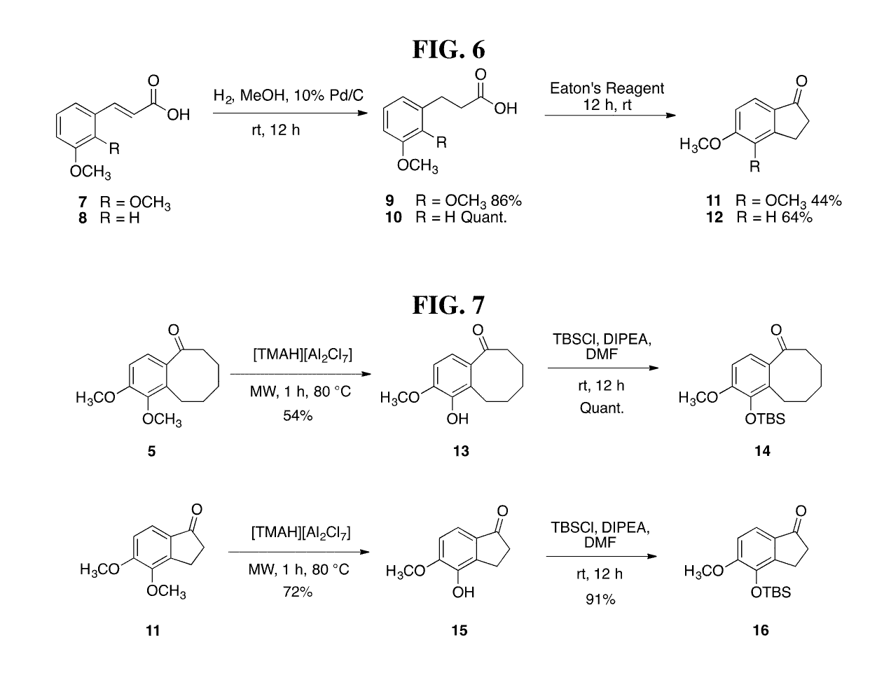

A similar catalytic reduction was utilized with starting material trans-2,3-dimethoxycinnamic acid to afford carboxylic acids 9 and 10, which were cyclized with Eaton's reagent to their corresponding indanone analogues 11 and 12, shown in Scheme 2 in FIG. 6. Benzocyclooctene 5 and indene 11 bearing the ortho dimethoxy motif were each subjected to a selective demethylation (Tanpure et al., 2013; Herdman et al., 2015; Kemperman, et al. 2003) to afford phenolic analogues 13 and 15, which were subsequently protected with TBS to yield 14 and 16, as shown in Scheme 3 in FIG. 7.

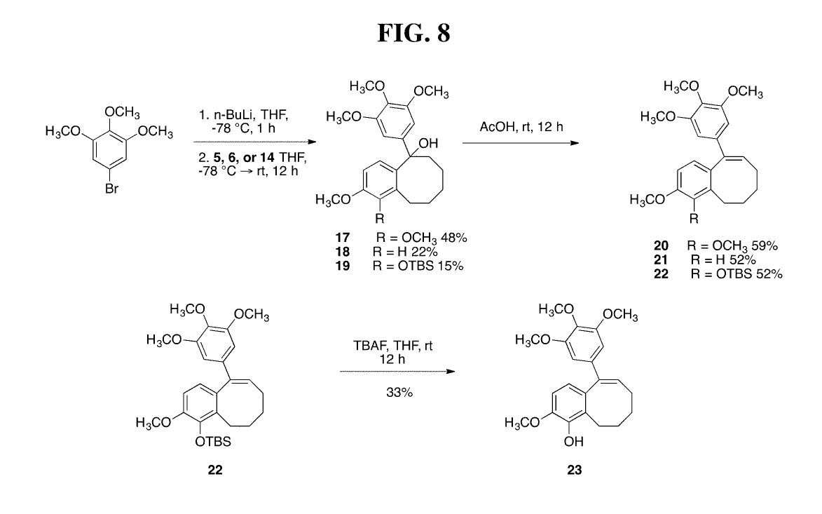

Trimethoxyphenyllithium (prepared from the corresponding aryl bromide) underwent a 1,2-addition reaction to benzocyclooctanone analogues 5, 6 and 14 to afford the corresponding tertiary alcohols 17, 18, and 19. Subsequent dehydration afforded benzocyclooctene analogues 20 and 21 and TBS-protected analogue 22, which underwent deprotection to afford phenolic benzocyclooctene 23, shown in Scheme 4 in FIG. 8.

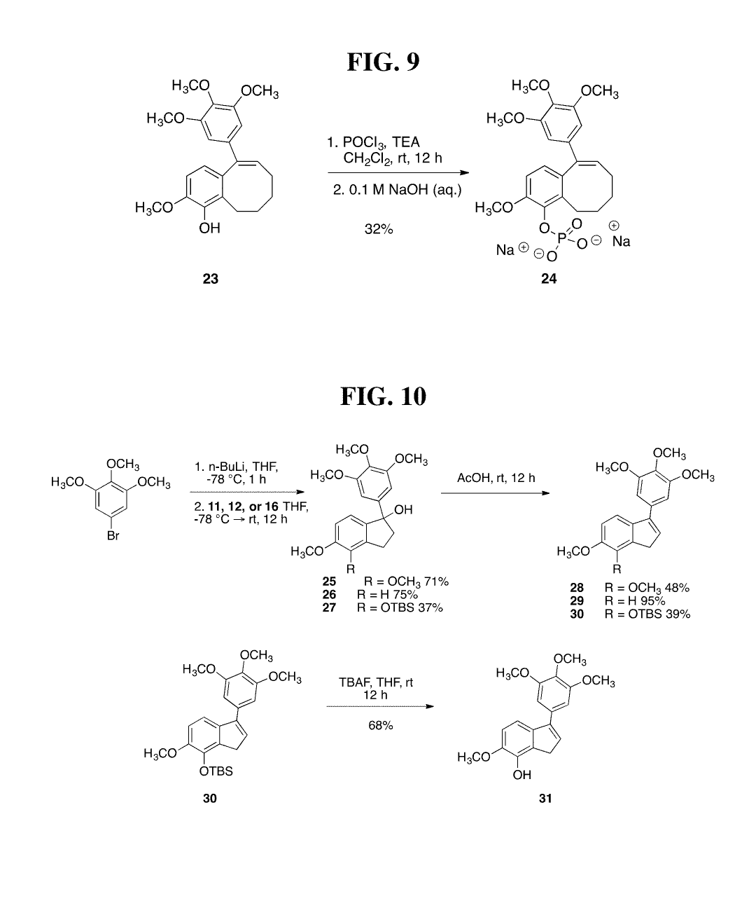

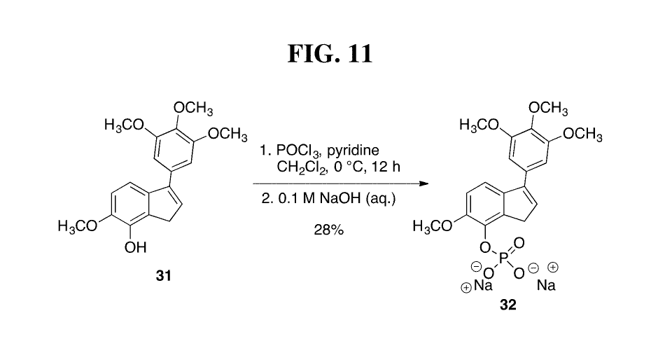

Phenolic analogue 23 was converted to its corresponding phosphate prodrug salt 24, in order to increase water solubility for in vivo studies, as shown in Scheme 5 in FIG. 9. Analogous aryl addition reactions were carried out on indanone intermediates 11, 12, and 16 to generate the corresponding tertiary alcohols 25, 26, and 27, as shown in Scheme 6 in FIG. 10. Subsequent dehydration afforded indene analogues 28 29, and 30. Removal of the TBS group afforded phenolic indene 31. The indene phenol 31 was also converted to its corresponding phosphate salt 32, as shown in Scheme 7 in FIG. 11. While TEA proved to be an acceptable base (due to ease of removal) for use in the synthesis of the eight membered ring phosphate salt 24, in the synthesis of the indene phosphate salt 32 TEA proved to be hard to remove during purification. Altering the base to pyridine solved this problem and allowed the water-soluble phosphate salt 32 to be synthesized in high purity.

Due to their increased size, eight membered rings are the first in the homologous series (3, 4, 5, 6, 7-membered rings) that can accommodate both Z and E double bond configurations. However, only the Z configurations were synthesized. X-ray crystal structures were obtained for benzocyclooctene analogues 20 and 23 to confirm their Z double bond configuration. FIG. 4 shows the structures of synthesized benzocyclooctene analogues 20, 21, 23, and 24 and indene analogues 28, 29, 31, and 32.

### Biological Evaluation

The four target benzocyclooctene and four indene analogues were evaluated for their cytotoxicity against human cancer cell lines and for their abilities to inhibit tubulin polymerization and colchicine binding to tubulin.
SRB Assay (Monks et al., 1991; Vichai et al., 2006)

Inhibition of growth of human cancer cells was assessed using the sulforhodamine B assay (SRB), as described in Monks et al., 1991. Cancer cell lines (DU-145, SK-OV-3, and NCI-H460) were plated at 7500-8000 cells/well into 96-well plates using DMEM supplemented with 5% fetal bovine serum/1% gentamicin sulfate and incubated for 24 h at 37° C. in a humidified incubator. Compound serial dilutions were then added. After 48 h treatment, the cells were fixed with trichloroacetic acid (10% final concentration), washed, dried, stained with sulforhodamine B dye (Acid red 52), washed to remove excess dye, and dried. SRB dye was solubilized, and absorbances were measured at wavelength 540 nm and normalized to values at wavelength 630 nm using an automated Biotek plate reader. A growth inhibition of 50% (GI₅₀ or the drug concentration causing 50% reduction in the net protein increase) was calculated from the absorbance data. Results are shown in Table 1 below.

Colchicine Binding Assay

Inhibition of [³H]colchicine binding to tubulin was measured using reaction mixtures (100 μL each) containing 1.0 μM tubulin, 5.0 μM [³H]colchicine (from Perkin-Elmer), 5% (v/v) dimethyl sulfoxide, potential inhibitors at 5.0 μM, and components that stabilize the colchicine binding activity of tubulin (Hamel et al., 1981) (1.0 M monosodium glutamate [adjusted to pH 6.6 with HCl in a 2.0 M stock solution], 0.5 mg/mL bovine serum albumin, 0.1 M glucose-1-phosphate, 1.0 mM MgCl₂, and 1.0 mM GTP). Incubation was for 10 min at 37° C., a time point selected because the binding reaction in control reaction mixtures is 40-60% complete. Reactions were stopped with 2.0 mL of ice-cold water, and the reaction mixtures were placed on ice. Each sample was poured onto a stack of two DEAE-cellulose filters (from Whatman), followed by 6 mL of ice-cold water. The samples were aspirated under reduced vacuum. The filters were washed three times with 2 mL water and placed into vials containing 5 mL of Biosafe II scintillation cocktail. Samples were counted 18 h later in a Beckman scintillation counter. Samples with inhibitors were compared to samples with no inhibitor, and percent inhibition was determined. All samples were corrected for the amount of radiolabel bound to the filters in the absence of tubulin. Results are shown in Table 1 below.

Inhibition of Tubulin Polymerization

Tubulin assembly experiments were performed in 0.25 mL reaction mixtures (final volume) (Hamel, 2003). The mixtures contained 1 mg/mL (10 μM) purified bovine brain tubulin, 0.8 M monosodium glutamate (pH 6.6), 4% (v/v) dimethyl sulfoxide, 0.4 mM GTP, and different compound concentrations. All reaction components except GTP were preincubated for 15 min at 30° C. in 0.24 mL. The mixtures were cooled to 0° C., and 10 μL of 10 mM GTP were added. Reaction mixtures were transferred to cuvettes held at 0° C. in Beckman DU-7400 and DU-7500 spectrophotometers equipped with electronic temperature controllers. The temperature was jumped to 30° C., taking about 30 s, and polymerization was followed at 350 nm for 20 min. The IC₅₀ was defined as the compound concentration that inhibited extent of assembly by 50% after 20 min. Results are shown in Table 1 below. Table 1 shows inhibition of tubulin polymerization [(expressed as half maximal inhibitory concentration (IC₅₀)] and cytotoxicity [expressed as growth inhibition of 50% (GI₅₀)] of the eight target analogues synthesized.

TABLE 1

| | Inhibition of | % Inhibition | | | |
|---|---|---|---|---|---|
| | tubulin | of | GI₅₀ (μM) SRB | | |
| | polymerization | colchicine | assay^{a} | | |
| Compound | IC₅₀ (μM) ± SD | binding ± SD | NCI-H460 | DU-145 | SK-OV-3 |
| CA4 | 1.0^{b} | 84 ± 3 (1 μM), | 0.00500^{c,d} | 0.00602^{c,d} | 0.00506^{d} |
| | | 98 ± 0.007 (5 μM) | | | |
| CA4P | >20^{b} | nr^{e} | 0.00282^{c} | 0.00336^{c} | 0.00190 |
| KGP18 | 1.4^{f} | r^{e} | 0.0000418^{g} | 0.0000249^{g} | 0.0000543^{g} |
| 20 | 4.5 ± 0.5 | 31 ± 0.6 (5 μM) | 0.395 | 0.448 | 0.512 |
| 21 | 3.0 ± 0.1 | 29 ± 5 (5 μM) | 0.431 | 0.570 | 0.400 |
| 23 | 1.2 ± 0.1 | 78 ± 4 (5 μM) | 0.107 | 0.105 | 0.0811 |
| 24 | >20 | nd^{h} | 0.0260 | 0.0410 | 0.0366 |
| 28 | >20 | nd^{h} | 42.2 | 34.6 | 9.46 |
| 29 | >20 | nd^{h} | 4.02 | 4.66 | 6.15 |
| 31 | 11 ± 2 | 17 ± 5 (5 μM) | 0.388 | 0.362 | 0.704 |
| 32 | >20 | nd^{h} | 1.50 | 3.34 | 0.334 |

^{a}Average of n ≥ 3 independent determinations
^{b}Data from Pettit et al., 2000.
^{c}For additional data, see Pettit et al., 2000.
^{d}Data from Devkota et. al., 2016
^{e}nr = not reported
^{f}For additional data, see Tanpure et al., 2012.
^{g}For additional data, see Sriram et al., 2008.
^{h}nd = not determined

Among the seven compounds synthesized and analyzed biologically, only the target benzocyclooctene analogues (20, 21, and 23) had activities as inhibitors of tubulin polymerization similar to those of CA4 and KGP18. Benzocyclooctene phenol 23 demonstrated the lowest IC₅₀ value (1.2 μM) among the compounds evaluated in this study. A binding study utilizing radiolabeled colchicine demonstrated that at a concentration 5 μM, phenol 23 inhibited colchicine binding by 78%, a twenty percent improvement when compared to CA4 (used as a positive control) in this assay. Considering the three indene analogues, only phenolic indene 31 demonstrated modest inhibition of tubulin polymerization, with an IC₅₀ value of 11 μM.

Benzocyclooctene phenol 23 and its corresponding phosphate prodrug salt 24 were the most cytotoxic analogues in these series against the three human cancer cell lines examined (for example, GI₅₀=0.105 and 0.0410 μM against DU-145 (prostate) for analogues 23 and 24, respectively). While structurally similar to KGP18, differing by the addition of one carbon to the aliphatic ring, the phenolic benzocyclooctene analogue is dramatically less cytotoxic than its benzosuberene counterpart. Compound 23 and 24 had GI₅₀ values in the submicromolar range, while the values obtained with KGP18 were all subnanomolar.

In Vivo Studies

Human breast cancer cells, MCF7-luc-GFP-mCherry (ATCC), were transfected sequentially with a lentivirus containing firefly luciferase reporter, GFP and mCherry reporter genes, as described in Liu et al., 2012. Highly expressing stable clones were isolated. Induction of tumors was carried out by injecting 10⁶ cells mixed with 50% Matrigel™ (BD Biosciences, San Jose, Calif.) into the right upper ventral mammary fat pads of female SCID-NOD mice (UTSW breeding colony). Tumors were allowed to grow to a size of 10-12 mm in diameter, determined by calipers, before selection for BLI. All animal procedures were approved by the University of Texas Southwestern Medical Center Institutional Animal Care and Use Committee.

Bioluminescence imaging was carried out as described in Liu et al., 2012. Briefly, anesthetized, tumor bearing mice (O₂, 2% isoflurane, Henry Schein Inc., Melville, N.Y.) were injected subcutaneously in the fore-back neck region with 80 μL of a solution of luciferase substrate, D-luciferin (sodium salt, 120 mg/kg, in saline, Gold Biotechnology, St. Louis, Mo.). Mice were maintained under anesthesia (2% isoflurane in oxygen, 1 dm³/min), while baseline bioluminescence imaging was performed using a Caliper Xenogen IVIS® Spectrum (Perkin-Elmer, Alameda, Calif.). A series of BLI images was collected over 35 min using the following settings: auto exposure time, f-stop=2, Field of view=D, binning=4 (medium). Light intensity-time curves obtained from these images were analyzed using Living Image® software and light emission compared based on area under the light emission curve. Mice were injected intraperitoneally with either 120 μL of saline (vehicle), CA4P (provided by Mateon Therapeutics, Inc.; 120 mg/kg in saline as used in Mason et al., 2011 or analogue 24 (120 mg/kg) in saline immediately after baseline BLI. Bioluminescence imaging was repeated, with new luciferin injections 4, 24, and 48 h later.

Bioluminescence imaging (BLI) is a highly valuable modality with diverse applications in a wide range of biological models and systems. BLI is a particularly useful tool for assessing and quantifying in vivo blood flow reduction following treatment with a VDA. In the current study MCF7 human breast cancer cells, which had been previously transfected stably to express the enzyme luciferase, as well as two fluorescent proteins, as designated by MCF7-luc-GFP-mCherry were implanted in SCID mice. Since the luciferase substrate luciferin can be delivered to the tumor via the blood stream, damage to the vessels by a VDA can be quantified by measurement of reduced bioluminescence upon injection of luciferin.

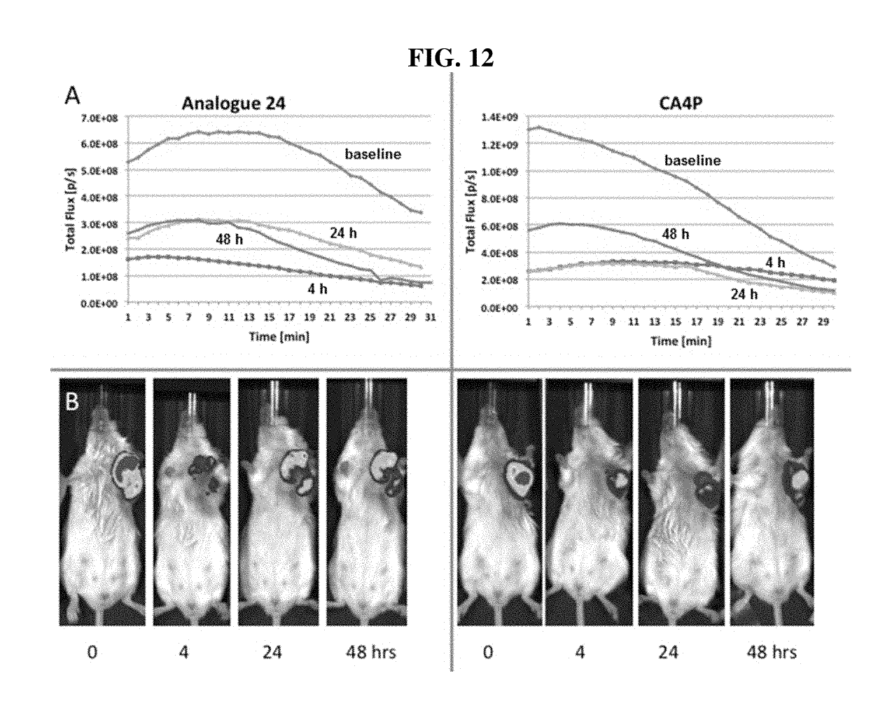

Three mice bearing the MCF7-luc-GFP-mCherry breast tumor were injected ip with analogue 24 (120 m/kg) with BLI assessment performed at baseline (0 h, pre-administration) and at 4 h, 24 h, and 48 h after administration of 24. In a contemporaneous study, one mouse was treated with CA4P (120 mg/kg) as a positive control and a separate mouse was treated with saline alone. A limited dose escalation study with analogue 24 in non-tumor bearing SCID mice (60-150 mg/kg, unpublished data) suggested that a dose of 120 mg/kg was tolerated and likely below the maximum tolerated dose (MTD), which has not yet been determined. The optimal CA4P dose in SCID mice for evaluation of VDA efficacy has been previously established at 120 mg/kg (Zhao et al., 2008). FIG. 12 shows dynamic bioluminescence with respect to vascular disruption. In FIG. 12A, graphs show evolution of light emission from individual MCF7-luc-GFP-mCherry breast tumors following administration of luciferin substrate subcutaneously in the fore-back region of each mouse at time 0. Respective curves are baseline, 4 h, 24 h and 48 h post administration of VDA. Left hand panel shows representative mouse with 120 mg/kg analogue 24 and right hand panel shows CA4P (120 mg/kg). FIG. 12B shows photographs of selected images at the 10 min. time point. Left hand group for analogue 24 and right hand group for CA4P for the same mice used to generate the curves for part A. Respective images are each scaled to same values.

Single dose treatment with both analogue 24 and CA4P resulted in a significant reduction in bioluminescence (approximately 80% decrease compared to baseline or saline control; p<0.05) at 4 h after compound injection. While bioluminescence intensity in the mouse treated with compound 24 recovered somewhat at 24 and 48 h, it remained significantly below baseline values (p<0.005). In this study, the BLI data for both analogue 24 and CA4P at 4 h were statistically different from the saline control, but not from each other or from the data obtained at other time points.

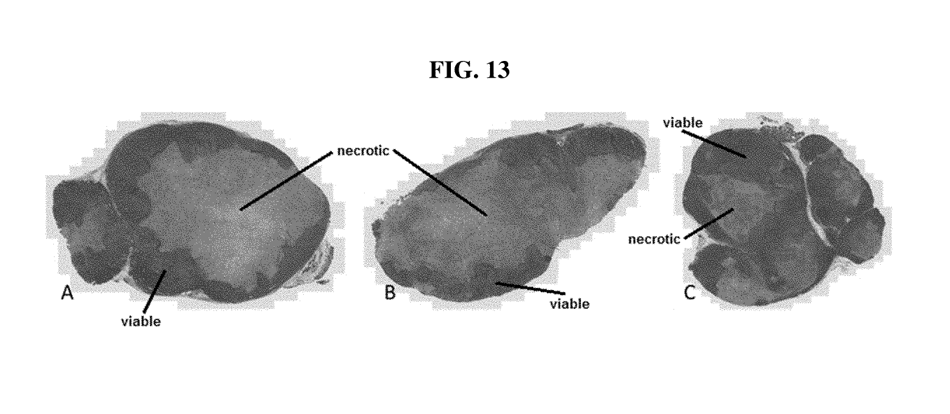

Following the 48 h BLI data acquisition, the tumors were excised, bisected and fixed in 4% paraformaldehyde solution. Tumor tissue was processed for paraffin embedding, sectioned and stained by routine methods. H&E staining was performed on one cross section from each tumor. Whole mount high resolution microscopy was obtained using a Zeiss Axioscan Z1 digital slide scanner. FIG. 13 shows histological assessment of tumor necrosis, with H&E stained tumor cross sections showing necrotic and viable regions at 48 h post treatment for (A) Analogue 24 (120 mg/kg), (B) CA4P (120 mg/kg), and (C) Saline. Routine staining using H&E indicated much more extensive necrosis throughout the tumors following administration of 24 or CA4P each at 120 mg/kg, as compared with saline control. Thus, analogue 24 may be a therapeutically useful VDA.

These studies, which focused on indene and benzocyclooctene ring systems, expanded the known SAR associated with the effect that fused aliphatic ring size plays in regard to cytotoxicity and inhibition of tubulin polymerization. From the group of eight analogues prepared by chemical synthesis with seven compounds evaluated biologically, three benzocyclooctene analogues were strong inhibitors of tubulin polymerization and one indene analogue was a modest inhibitor. The most promising compound (benzocyclooctene analogue 23) was prepared and evaluated as its corresponding water-soluble phosphate salt 24. It demonstrated significant in vivo reduction of blood flow (as evidenced by BLI) in an MCF7-luc-GFP-mCherry tumor model in SCID mice, and the results indicated that 24 appears promising as a VDA for potential cancer treatment.

### Synthesis and Data

Tetrahydrofuran (THF), CH₂Cl₂, ethanol, methanol, dimethylformamide (DMF), and acetonitrile were used in their anhydrous forms. Reactions were performed under nitrogen gas. Thin-layer chromatography (TLC) plates (precoated glass plates with silica gel 60 F254, 0.25 mm thickness) were used to monitor reactions. Purification of intermediates and products was carried out with a Biotage Isolera flash purification system using silica gel (200-400 mesh, 60 Å). Intermediates and products synthesized were characterized on the basis of their ¹H NMR (500 or 600 MHz) and ¹³C NMR (125 or 150 MHz) spectroscopic data using a Varian VNMRS 500 MHz or a Bruker Ascend 600 MHz instrument. Spectra were recorded in CDCl₃ and D₂O. All chemical shifts are expressed in ppm (δ), coupling constants (J) are presented in Hz, and peak patterns are reported as singlet (s), doublet (d), triplet (t), quartet (q), pentet (p), septet (sept), and multiplet (m).

Purity of the target compounds was further analyzed at 25° C. using an Agilent 1200 HPLC system with a diode-array detector (λ=190-400 nm), a Zorbax XDB-C18 HPLC column (4.6 mm Åμ150 mm, 5 μm), and a Zorbax reliance cartridge guard-column; method A: solvent A, acetonitrile, solvent B, H₂O; gradient, 10% A/90% B to 100% A/0% B or method B: solvent A, acetonitrile, solvent B, 0.1% TFA in H₂O over 0 to 40 min; post-time 10 min; flow rate 1.0 mL/min; injection volume 20 μL; monitored at wavelengths of 210, 254, 230, 280, and 360 nm. Mass spectrometry was carried out under positive ESI (electrospray ionization) using a Thermo Scientific LTQ Orbitrap Discovery instrument.

### 6-(2′,3′-Dimethoxyphenyl)hex-5-enoic acid (1) (WO2012/068284)

To a well-stirred solution of 4-(carboxybutyl)triphenyl phosphonium bromide (13.47 g, 30.39 mmol) dissolved in THF (500 mL) at rt was added potassium tert-butoxide (7.43 g, 66.2 mmol). After 1 h, 2,3-dimethoxybenzaldehyde (5.02 g, 30.1 mmol) dissolved in THF (100 mL) was added to the original reaction mixture, and stirring at room temperature was continued. After 12 h, the THF was evaporated under reduced pressure, and the resulting material was quenched with 2 M HCl (100 mL) and extracted with EtOAc (2×100 mL). The combined organic layers were evaporated under reduced pressure. Purification by flash column chromatography using a pre-packed 100 g silica column [solvent A: EtOAc; solvent B: hexanes; gradient: 10% A/90% B (1 CV), 10% A/90% B→50% A/50% B (10 CV), 50% A/50% B (2 CV); flow rate: 40 mL/min; monitored at 254 and 280 nm] afforded compound 1 (3.61 g, 14.4 mmol, 48%) as a yellow oil. NMR characterization was determined after the next step.

### 6-(2′,3′-Dimethoxyphenyl)hexanoic acid (3) (WO2012/068284)

To a well-stirred solution of carboxylic acid 1 (3.61 g, 14.4 mmol) dissolved in MeOH (150 mL) was added 10% Pd on carbon (0.74 g) and H₂ gas (balloon), and the reaction was stirred at room temperature for 12 h. The reaction mixture was then filtered through Celite® (Sigma-Aldrich diatomaceous earth filter), the Celite® was washed with EtOAc (3×50 mL), and the filtrate (MeOH and EtOAc) was evaporated under reduced pressure. The organic material was purified by flash chromatography using a pre-packed 100 g silica column [solvent A: EtOAc; solvent B: hexanes; gradient: 7% A/93% B (1 CV), 7% A/93% B→40% A/60% B (10 CV), 40% A/60% B (2 CV); flow rate: 40 mL/min; monitored at 254 and 280 nm] to afford carboxylic acid 3 (3.63 g, 14.4 mmol, quantitative) as a colorless oil. ¹H NMR (CDCl₃, 600 MHz) δ 11.83 (1H, s), 7.01 (1H, t, J=9.5 Hz), 6.80 (2H, J=10 Hz), 3.88 (3H, s), 3.86 (3H, s), 2.68 (2H, t, J=9 Hz), 2.39 (2H, t, J=16 Hz), 1.70 (4H, m), 1.46 (2H, p, J=9.5 Hz). ¹³C NMR (CDCl₃, 150 Hz) δ 180.1, 152.7, 147.1, 136.2, 123.8, 121.9, 110.2, 60.5, 55.5, 34.0, 30.4, 29.6, 28.9, 24.5.

### 1,2-Dimethoxy-benzocylcooct-5-one (5) (WO2012/068284)

To carboxylic acid 3 (4.40 g, 17.4 mmol) was added Eaton's reagent (35 mL, 3 g per mmol of compound 3), and the mixture was stirred at room temperature for 12 h, at which time it was poured over ice, which was allowed to melt, and the solution was neutralized with sodium bicarbonate. The organic layer was extracted with EtOAc (3×50 mL), dried over sodium sulfate, evaporated under reduced pressure, and purified by flash chromatography using a pre-packed 100 g silica column [solvent A: EtOAc; solvent B: hexanes; gradient: 7% A/93% B (1 CV), 7% A/93% B→40% A/60% B (10 CV), 40% A/60% B (2 CV); flow rate: 40 mL/min; monitored at 254 and 280 nm] to afford ketone 5 (0.58 g, 2.5 mmol, 14%) as a yellow oil. ¹H NMR (CDCl₃, 600 MHz) δ 7.35 (1H, d, J=8.4 Hz), 6.65 (1H, d, J=9 Hz), 3.69 (3H, s), 3.59 (3H, s), 2.94 (2H, t, J=6.6 Hz), 2.73 (2H, t, J=6.6 Hz), 1.59 (4H, m), 1.27 (2H, p, J=6.6 Hz). ¹³C NMR (CDCl₃, 150 Hz) δ 204.9, 155.5, 146.2, 134.7, 133.5, 124.8, 109.6, 60.7, 55.6, 43.9, 27.0, 25.4, 24.7, 24.1.

### 4.1.4. [TMAH][Al₂Cl₇] (Kemperman et al., 2003)

To dry CH₂Cl₂ (150 mL) was added AlCl₃ (19.84 g, 149.08 mmol), which was stirred and cooled to 0° C. Trimethylamine hydrochloride (7.11 g, 74.54 mmol) was added, and the mixture was stirred for 2 h at room temperature. The resulting liquid was stored at room temperature under nitrogen.

### 1-Hydroxy-2-methoxy-benzocyclooct-5-one (13)

To ketone 5 (0.57 g, 2.3 mmol) in a 20 mL microwave vial was added [TMAH][Al₂Cl₇] (7.54 mL, 4.69 mmol), and the mixture was reacted in a microwave for 1 h at 80° C. The solution was poured into water (50 mL), extracted with EtOAc (3×25 mL), dried over sodium sulfate, and evaporated under reduced pressure. The crude reaction mixture was purified by flash chromatography using a pre-packed 50 g silica column [solvent A: EtOAc; solvent B: hexanes; gradient: 7% A/93% B (1 CV), 7% A/93% B→60% A/40% B (10 CV), 60% A/40% B (2 CV); flow rate: 50 mL/min; monitored at 254 and 280 nm] affording phenol 13 (0.28 g, 1.3 mmol, 54%) as a clear oil. ¹H NMR (CDCl₃, 600 MHz) δ 7.23 (1H, d, J=8.4 Hz), 6.73 (1H, d, J=8.4 Hz), 6.11 (1H, s) 3.85 (3H, s), 3.06 (2H, t, J=6.6 Hz), 2.88 (2H, t, J=6.6 Hz), 1.75 (4H, m), 1.49 (2H, p, J=6 Hz). ¹³C NMR (CDCl₃, 150 Hz) δ 206.4, 148.7, 142.9, 133.7, 126.7, 120.1, 107.9, 56.0, 44.4, 25.9, 25.7, 25.3, 23.9.

### 1-((tert-Butyldimethylsilyl)oxy)-2-methoxy-benzocyclooct-5-one (14)

Phenol 13 (0.22 g, 1.0 mmol) was dissolved in DMF (50 mL). TBSCl (0.30 g, 2.0 mmol) and DIPEA (0.52 mL, 3.0 mmol) were added, and the reaction was stirred for 12 h at room temperature. The reaction mixture was washed with water (50 mL), extracted with EtOAc (5×30 mL), dried over sodium sulfate, and evaporated under reduced pressure. The crude reaction mixture was purified by flash chromatography using a pre-packed 25 g silica column [solvent A: EtOAc; solvent B: hexanes; gradient: 2% A/98% B (1 CV), 2% A/98% B→20% A/80% B (10 CV), 20% A/80% B (2 CV); flow rate: 50 mL/min; monitored at 254 and 280 nm] affording TBS-protected ketone 14 (0.31 g, 0.93 mmol, quantitative) as a white solid. ¹H NMR (CDCl₃, 600 MHz) δ 7.04 (1H, d, J=8.4 Hz), 6.56 (1H, d, J=9 Hz), 3.62 (3H, s), 2.87 (2H, t, J=6 Hz), 2.69 (2H, t, J=7.2 Hz), 1.58 (4H, m), 1.33 (2H, p, J=3 Hz), 0.81 (9H, s), 0.00 (6H, s). ¹³C NMR (CDCl₃, 150 Hz) δ 207.1, 151.9, 141.9, 133.7, 131.5, 120.9, 108.5, 54.5, 44.5, 26.2, 26.0, 25.8, 25.5, 23.7, 18.8, −3.9.

### 1-((tert-Butyldimethylsilyl)oxy)-2-methoxy-5-(3′,4′,5′-trimethoxyphenyl)-benzocyclooctan-5-ol (19)

To an oven dried flask containing THF (50 mL) was added 3,4,5-trimethoxyphenyl bromide (0.20 g, 0.81 mmol), and the solution was cooled to −78° C. n-BuLi (1.3 mL, 0.85 mmol) was slowly added to the reaction mixture, which was then stirred at −78° C. for 1 h. TBS-protected 14 (0.20 g, 0.60 mmol) was added dropwise to the flask, and the reaction mixture was stirred while warming from −78° C. to room temperature over 12 h, at which time the reaction mixture was washed with water, extracted with EtOAc (3×50 mL), dried over sodium sulfate, and evaporated under reduced pressure. The organic material was purified by flash chromatography using a pre-packed 25 g silica column [solvent A: EtOAc; solvent B: hexanes; gradient: 2% A/98% B (1 CV), 2% A/98% B→20% A/80% B (10 CV), 20% A/80% B (2 CV); flow rate: 50 mL/min; monitored at 254 and 280 nm] affording tertiary alcohol 19 (0.044 g, 0.088 mmol, 15%) as a yellow oil. NMR characterization was performed after the next step.

### 1-((tert-Butyldimethylsilyl)oxy)-2-methoxy-5-(3′,4′,5′-trimethoxyphenyl)benzocyclooct-5-ene (22)

Acetic acid (10 mL) was added to tertiary alcohol 19 (0.044 g, 0.088 mmol), and the reaction mixture was stirred for 12 h at room temperature, at which time the mixture was washed with water (50 mL), extracted with EtOAc (3×30 mL), and dried over sodium sulfate. The organic phase was evaporated under reduced pressure, and the crude reaction mixture was purified by flash chromatography using a pre-packed 10 g silica column [solvent A: EtOAc; solvent B: hexanes; gradient: 2% A/98% B (1 CV), 2% A/98% B→20% A/80% B (10 CV), 20% A/80% B (2 CV); flow rate: 12 mL/min; monitored at 254 and 280 nm] affording TBS-protected 22 (0.022 g, 0.045 mmol, 52%) as a clear oil. ¹H NMR (CDCl₃, 600 MHz) δ 6.68 (1H, d, J=8.4 Hz), 6.56 (1H, d, J=8.4 Hz), 6.42 (2H, s), 6.19 (1H, t, J=7.8 Hz), 3.84, (3H, s), 3.80 (3H, s), 3.78 (6H, s), 3.27 (1H, overlapping doublets, J=12.6 Hz), 2.23 (1H, m), 1.96 (1H, m), 1.75 (1H, m), 1.66 (1H, q, J=22 Hz), 1.37 (2H, m), 1.02 (9H, s), 0.27 (3H, s), 0.24 (3H, s).

### 1-Hydroxy-2-methoxy-5-(3′,4′,5′-trimethoxyphenyl)-benzocyclooct-5-ene (23) (WO2012/068284)

TBS-protected benzocyclooctene 22 (0.022 g, 0.045 mmol) was dissolved in THF (10 mL), TBAF (0.031 g, 0.099 mmol) was added, and the reaction mixture was stirred at room temperature for 12 h. The solution was washed with water (50 mL), extracted with EtOAc (3×30 mL), dried over sodium sulfate, and evaporated under reduced pressure. The crude reaction mixture was purified by flash chromatography using a pre-packed 10 g silica column [solvent A: EtOAc; solvent B: hexanes; gradient: 3% A/97% B (1 CV), 3% A/97% B→30% A/70% B (10 CV), 30% A/70% B (2 CV); flow rate: 36 mL/min; monitored at 254 and 280 nm] affording benzocyclooctene 23 (0.0053 g, 0.014 mmol, 33%) as a white solid. ¹H NMR (CDCl₃, 600 MHz) δ 6.70 (1H, d, J=8.4 Hz), 6.53 (1H, d, J=8.4 Hz), 6.43 (2H, s), 6.21 (1H, t, J=8.4 Hz), 5.76 (1H, s), 3.92 (3H, s), 3.84 (3H, s), 3.78 (6H, s), 3.25 (1H, dd, J=12.6, 7.8 Hz), 2.30 (2H, m), 2.03 (1H, m), 1.79 (1H, m), 1.68 (1H, dt, J=22.2, 11.4 Hz), 1.45 (1H, qd, J=13.2, 4.8 Hz), 1.34 (1H, qd, J=13.2, 4.8 Hz)¹³C NMR (CDCl₃, 150 MHz) δ 152.8, 145.3, 142.7, 139.7, 139.1, 137.1, 132.3, 129.8, 129.7, 120.3, 107.8, 104.6, 60.9, 56.1, 56.0, 28.3, 26.5, 25.8, 24.7. HRMS: Obsvd 393.1693 [M+Na⁺], Calcd for C₂₂H₂₆O₅Na: 393.1672. HPLC: 16.61 min.

### Sodium 2-methoxy-5-(3′,4′,5′-trimethoxyphenyl)-benzocyclooct-5-en-1-yl phosphate (24)

Phosphorus oxychloride (0.18 mL, 1.9 mmol) was cooled to 0° C. in DCM (10 mL) and triethylamine (0.68 mL, 4.9 mmol) was added, and the reaction mixture was stirred for 5 min. Benzocyclooctene 23 (0.14 g, 0.38 mmol) in DCM (5 mL) was added to the reaction dropwise, and the reaction mixture was stirred at 0° C. for 1 h and then warming to room temperature over 12 h. The mixture was then evaporated under reduced pressure. DCM (10 mL) was added to the resulting residue, and the resulting solution was again evaporated under reduced pressure. This was repeated two more times. The resulting solid was dissolved in a mixture of THF and water (2:1, 6 mL total) and stirred for 1 h. The solution was then cooled to 0° C., and 0.1 M NaOH was added until a pH of 10 was achieved. The solution was then evaporated under reduced pressure, and the crude product was purified by a C18 30 g reversed phase column [solvent A: acetonitrile; solvent B: water; gradient: 10% A/90% B (1 CV), 10% A/90% B→100% A/0% B (10 CV), 100% A/0% B (2 CV); flow rate: 25 mL/min; monitored at 254 and 280 nm] to afford phosphate salt 24 (0.06 g, 0.12 mmol, 32%) as a brown solid. ¹H NMR (D₂O, 600 MHz) δ 6.69 (1H, d, J=8.4 Hz), 6.52 (2H, s), 6.47 (1H, d, J=8.4 Hz), 6.20 (1H, t, J=7.4 Hz), 3.72 (3H, s), 3.67 (6H, s), 3.63 (3H, s), 3.45 (1H, dd, J=13.2, 8.4 Hz), 2.14 (1H, dt, J=13.8, 8.4), 2.05 (1H, t, J=12 Hz), 1.91 (1H, m), 1.63 (1H, m), 1.43 (1H, dt, J=22.2, 12 Hz), 1.30 (1H, qd, J=13.2, 4.8 Hz), 1.16 (1H, qd, J=13.2, 4.8 Hz). ¹³C NMR (D₂O, 150 MHz) δ 152.2, 151.4 (d, J=2.25 Hz), 141.1 (d, J=6.75 Hz), 140.0, 138.8, 138.0 (d, J=3.38 Hz), 135.8, 131.5, 131.2, 123.8, 109.5, 104.9, 60.9, 56.0, 55.6, 28.1, 26.6, 26.3, 23.8. ³¹P NMR (D₂O, 242 MHz) δ −0.25. HRMS: Obsvd 495.1249 [M+H], Calcd for C₂₂H₂₆O₈Na₂P⁺: 495.1155 HPLC: 5.46 min.

### 1,2-Dimethoxy-5-(3′,4′,5′-trimethoxyphenyl)-benzocyclooctan-5-ol (17)

To an oven dried flask of THF (50 mL) was added 3,4,5-trimethoxyphenyl bromide (0.26 g, 1.0 mmol), and the solution was cooled to −78° C. n-BuLi (0.44 mL, 1.1 mmol) was slowly added to the reaction mixture, which was then stirred at −78° C. for 1 h. Benzocyclooctone 5 (0.18 g, 0.77 mmol) was added dropwise to the flask, and the reaction was stirred while warming from −78° C. to room temperature over 12 h, at which time the reaction mixture was washed with water (50 mL), extracted with EtOAc (3×50 mL), dried over sodium sulfate, and evaporated under reduced pressure. The crude product was purified by flash chromatography using a pre-packed 25 g silica column [solvent A: EtOAc; solvent B: hexanes; gradient: 7% A/93% B (1 CV), 7% A/93% B→60% A/40% B (10 CV), 60% A/40% B (2 CV); flow rate: 50 mL/min; monitored at 254 and 280 nm] affording tertiary alcohol 17 (0.15 g, 0.37 mmol, 48%) as a clear oil. NMR characterization was performed after the next step.

### 1,2-Dimethoxy-5-(3′,4′,5′-trimethoxyphenyl)-benzocyclooct-5-ene (20)

Acetic acid (20 mL) was added to tertiary alcohol 17 (0.15 g, 0.37 mmol), and the reaction mixture was stirred for 12 h at room temperature, at which time the mixture was washed with water (50 mL), extracted with EtOAc (3×30 mL), and dried over sodium sulfate. The organic phase was evaporated under reduced pressure, and the crude organic product was purified by flash chromatography using a pre-packed 10 g silica column [solvent A: EtOAc; solvent B: hexanes; gradient: 7% A/93% B (1 CV), 7% A/93% B→60% A/40% B (10 CV), 60% A/40% B (2 CV); flow rate: 12 mL/min; monitored at 254 and 280 nm]affording benzocyclooctene 20 (0.082 g, 0.213 mmol, 59%) as a white solid. ¹H NMR (CDCl₃, 600 MHz) δ 6.74 (1H, d, J=8.5 Hz), 6.72 (1H, d, J=8 Hz), 6.41 (2H, s), 6.21 (1H, dd, J=10.8, 9 Hz), 3.93 (3H, s), 3.88 (3H, s), 3.84 (3H, s), 3.78 (6H, s), 3.24 (1H, dd, J=15, 9.6 Hz), 2.27 (2H, m), 2.06 (1H, m), 1.77 (1H, m), 1.65 (1H, dt, J=25.2, 12.6 Hz), 1.37 (2H, m). ¹³C NMR (CDCl₃, 150 MHz) δ 152.8, 151.8, 146.3, 139.7, 139.0, 137.5, 137.2, 131.9, 129.7, 124.8, 109.5, 104.7, 60.9, 60.7, 56.1, 55.6, 28.3, 27.8, 26.0, 24.6. HRMS: Obsvd 407.1859 [M+Na⁺], Calcd for C₁₉H₂₈O₂Na: 407.1829. HPLC: 18.53 min.

### 6-(3′-Methoxyphenyl)hex-5-enoic acid (2) (Gapinski et al., 1990)

To a well-stirred solution of 4-(carboxybutyl)triphenyl phosphonium bromide (16.29 g, 36.75 mmol) dissolved in THF (500 mL) at rt was added potassium tert-butoxide (9.09 g, 81.0 mmol). After 1 h, 2,3-dimethoxybenzaldehyde (4.47 mL, 36.8 mmol) dissolved in THF (100 mL) was added to the original reaction mixture, and the resulting reaction mixture was stirred at room temperature for 12 h. The THF was evaporated under reduced pressure, and the resulting material was quenched with 2 M HCl (100 mL) and extracted with EtOAc (2×100 mL). The combined organic layer was evaporated under reduced pressure and purified by flash chromatography using a pre-packed 100 g silica column [solvent A: EtOAc; solvent B: hexanes; gradient: 10% A/90% B (1 CV), 10% A/90% B→80% A/20% B (10 CV), 80% A/20% B (2 CV); flow rate: 40 mL/min; monitored at 254 and 280 nm] affording carboxylic acid 2 (8.01 g, 36.7 mmol, 99%) as a yellow oil. NMR characterization was performed after the next step.

### 6-(3′-Methoxyphenyl)hexanoic acid (4) (Ross et al., 1965; Bauer et al., 2013)

To dissolved carboxylic acid 2 (8.01 g, 36.7 mmol) in MeOH (150 mL) was added 10% Pd on carbon (0.46 g) and H₂ gas (balloon). The reaction mixture was stirred at room temperature for 12 h. The mixture was then filtered through Celite®, the Celite® was washed with EtOAc (3×50 mL), and the filtrate (MeOH and EtOAc) was evaporated under reduced pressure. The combined organic material was purified by flash chromatography using a pre-packed 100 g silica column [solvent A: EtOAc; solvent B: hexanes; gradient: 7% A/93% B (1 CV), 7% A/93% B→60% A/40% B (10 CV), 60% A/40% B (2 CV); flow rate: 40 mL/min; monitored at 254 and 280 nm] to afford carboxylic acid 4 (7.53 g, 33.9 mmol, 93%) as a colorless oil. ¹H NMR (CDCl₃, 600 MHz) δ 7.21 (1H, t, J=10.2 Hz), 6.78 (1H, d, J=9.6 Hz), 6.76 (2H, m), 3.81 (3H), 2.62 (2H, t, J=9 Hz), 2.38 (2H, t, J=9 Hz), 1.67 (4H, overlapping pentets, J=9 Hz), 1.41 (2H, p, J=9.6 Hz).

### 2-Methoxy-benzocyclooct-5-ene (6) (Eaton et al., 1973; Bauer et al., 2013; Mandal et al., 1988; Kammath et al., 2013)

Carboxylic acid 4 (7.53 g, 33.9 mmol) was dissolved in CH₂Cl₂ (100 mL) and cooled to 0° C. Eaton's reagent (68 mL, 3 g per mmol of compound 4) was added, and the mixture was stirred while warming to room temperature over 12 h, at which time it was poured over ice, which was allowed to melt, and the solution was neutralized with NaHCO₃. The organic layer was extracted with EtOAc (3×50 mL), dried over sodium sulfate, evaporated under reduced pressure, and purified by flash chromatography using a pre-packed 100 g silica column [solvent A: EtOAc; solvent B: hexanes; gradient: 5% A/95% B (1 CV), 5% A/95% B→40% A/60% B (10 CV), 40% A/60% B (2 CV); flow rate: 40 mL/min; monitored at 254 and 280 nm] to afford ketone 6 (0.45 g, 2.2 mmol, 7%) as a yellow oil. ¹H NMR (CDCl₃, 600 MHz) δ 7.81 (1H, d, J=9 Hz), 6.63 (1H, dd, J=9, 2.4 Hz), 6.51 (1H, d, J=3 Hz), 3.65 (3H, s), 2.94 (2H, t, J=6.6 Hz), 2.81 (2H, t, J=7.2 Hz), 1.67 (2H, p, J=7.2 Hz), 1.61 (2H, p, J=6.6 Hz), 1.26 (2H, p, J=6 Hz). ¹³C NMR (CDCl₃, 150 Hz) δ 202.4, 162.8, 143.1, 132.2, 131.4, 116.5, 111.5, 55.1, 42.6, 35.3, 27.6, 24.5, 23.0.

### 2-Methoxy-5-(3′,4′,5′-trimethoxyphenyl)-benzocyclooctan-5-ol (18)

To an oven dried flask containing THF (50 mL) was added 3,4,5-trimethoxyphenyl bromide (0.73 g, 3.0 mmol), and the solution was cooled to −78° C. n-BuLi (4.9 mL, 3.1 mmol) was slowly added to the reaction mixture, which was then stirred at −78° C. for 1 h. Ketone 6 (0.45 g, 2.2 mmol) was then added dropwise to the flask, and the reaction mixture was stirred while warming from −78° C. to room temperature over 12 h, at which time the reaction mixture was washed with water, extracted with EtOAc (3×50 mL), dried over sodium sulfate, and evaporated under reduced pressure. The crude reaction mixture was purified by flash chromatography using a pre-packed 25 g silica column [solvent A: EtOAc; solvent B: hexanes; gradient: 7% A/93% B (1 CV), 7% A/93% B→60% A/40% B (10 CV), 60% A/40% B (2 CV); flow rate: 50 mL/min; monitored at 254 and 280 nm] to afford tertiary alcohol 18 (0.18 g, 0.48 mmol, 22%) as a yellow oil. NMR characterization was performed after the next step.

### 2-Methoxy-5-(3′,4′,5′-trimethoxyphenyl)-benzocyclooct-5-ene (21)

Acetic acid (10 mL) was added to tertiary alcohol 18 (0.18 g, 0.48 mmol), and the reaction mixture was stirred for 12 h at rt, at which time the mixture was washed with water (50 mL), extracted with EtOAc (3×30 mL), and dried over sodium sulfate. The organic phase was evaporated under reduced pressure, and the crude reaction mixture was purified by flash chromatography using a pre-packed 10 g silica column [solvent A: EtOAc; solvent B: hexanes; gradient: 5% A/95% B (1 CV), 5% A/95% B→40% A/60% B (10 CV), 40% A/60% B (2 CV); flow rate: 12 mL/min; monitored at 254 and 280 nm] to afford benzocyclooctene 21 (0.089 g, 0.25 mmol, 52%) as a white solid. ¹H NMR (CDCl₃, 600 MHz) δ 6.95 (1H, d, J=8.4 Hz), 6.85 (1H, d, J=2.4 Hz), 6.73 (1H, dd, J=6.4, 2.4 Hz), 6.44 (2H, s), 6.24 (1H, dd, J=9, 7.8 Hz), 3.863 (3H, s), 3.857 (3H, s), 3.80 (6H, s), 2.83 (1H, dd, J=13.2, 7.8 Hz), 2.56 (1H, t, J=12.6 Hz), 2.29 (1H, dt, J=13.8, 7.8 Hz), 2.07 (1H, m), 1.80 (1H, m) 1.64 (1H, dt, J=21, 10.8 Hz), 1.41 (1H, qd, J=12.6, 4.8 Hz), 1.34 (1H, qd, J=13.2, 5.4 Hz). ¹³C NMR (CDCl₃, 150 Hz) δ 159.0, 152.8, 144.6, 139.7, 139.0, 137.1, 130.7, 129.3, 114.0, 111.4, 104.6, 60.9, 56.1, 55.2, 33.5, 28.8, 28.3, 24.9. HRMS: Obsvd 377.1724 [M+Na⁺], Calcd for C₂₂H₂₆O₄Na: 377.1723. HPLC: 19.29 min.

### 3-(2′,3′-Dimethoxyphenyl)propanoic acid (9) (Perkin et al., 1914; Williams et al., 2014)

Trans-2,3-dimethoxycinnamic acid (7) (5.00 g, 24.0 mmol) was dissolved in MeOH (100 mL), 10% Pd on carbon (0.82 g) was added, and the mixture was stirred for 12 h under H₂ (balloon). The mixture was then filtered through Celite®, the Celite® was washed with EtOAc (2×50 mL). The organic layer (EtOAc and MeOH) was dried over sodium sulfate, concentrated and purified by flash chromatography using a prepacked 100 g silica column [solvent A: EtOAc; solvent B: hexane; gradient: 7% A/93% B (1 CV), 7% A/93% B→60% A/40% B (10 CV), 60% A/40% B (2 CV); flow rate: 40 mL/min; monitored at 254 nm and 280 nm] to afford carboxylic acid 9 (4.34 g, 20.6 mmol, 86%) as a white solid. ¹H NMR (CDCl₃, 500 MHz) δ 11.91 (1H, s), 6.97 (1H, t, J=8 Hz), 6.78 (2H, d, J=8 Hz), 3.85 (3H, s), 3.80 (3H, s), 2.98 (2H, t, J=7.5 Hz), 2.67 (2H, t, J=7.5 Hz). ¹³C NMR (CDCl₃, 150 MHz) δ 179.4, 152.7, 147.1, 133.9, 124.0, 121.7, 111.0, 60.4, 55.5, 34.7, 25.3.

### 4,5-Dimethoxy-2,3-dihydro-1H-inden-1-one (11) (Perkin et al., 1914; Pati et al., 2015)

Carboxylic acid 9 (4.99 g, 23.7 mmol) was mixed with Eaton's reagent (47.5 mL, 3 g per mmol of carboxylic acid 9) and stirred for 72 h at room temperature. The mixture was then poured over ice, neutralized, and extracted with EtOAc (3×75 mL). The organic layer was dried over sodium sulfate, concentrated and purified by flash chromatography using a prepacked 100 g silica column [solvent A: EtOAc; solvent B: hexane; gradient: 7% A/93% B (1 CV), 7% A/93% B→60% A/40% B (10 CV), 60% A/40% B (2 CV); flow rate: 40 mL/min; monitored at 254 nm and 280 nm] to afford ketone 11 (2.01 g, 10.5 mmol, 44%) as a yellow solid. ¹H NMR (CDCl₃, 600 MHz) δ 7.06 (1H, d, J=8.4 Hz), 6.62 (1H, d, J=8.4 Hz), 3.61 (3H, s), 3.57 (3H, s), 2.70 (2H, t, J=5.4 Hz), 2.25 (2H, t, J=5.4 Hz). ¹³C NMR (CDCl₃, 150 MHz) δ 204.6, 157.1, 147.4, 145.0, 130.7, 119.4, 112.0, 59.8, 55.8, 36.0, 22.1.

### 4-Hydroxy-5-methoxy-2,3-dihydro-1H-inden-1-one (15) (Kemperman et al., 2003; Fujii et al., 1977; Day et al., 2011)

Ketone 11 (0.70 g, 3.2 mmol) was added to a 20 mL microwave vial with [TMAH][Al₂Cl₇] (10.0 mL, 7.26 mmol) and microwaved for 1 h at 80° C. The mixture was poured into water, extracted with CH₂Cl₂ (3×30 mL), dried over sodium sulfate and purified by flash chromatography using a prepacked 50 g silica column [solvent A: EtOAc; solvent B: hexane; gradient: 12% A/88% B (1 CV), 12% A/88% B→100% A/0% B (10 CV), 100% A/0% B (2 CV); flow rate: 50 mL/min; monitored at 254 nm and 280 nm] to afford phenol 15 (0.42 g, 2.36 mmol, 72%) as a brown solid. ¹H NMR (CDCl₃, 600 MHz) δ 7.35 (1H, d, J=10.2 Hz), 6.92 (1H, d, J=10.2 Hz), 5.81 (1H, s), 3.97 (3H, s), 3.07 (2H, t, J=6.6 Hz), 2.69 (2H, t, J=6.6 Hz). ¹³C NMR (CDCl₃, 150 MHz) δ 206.0, 150.9, 142.1, 140.5, 131.4, 116.1, 110.4, 56.4, 36.5, 21.9.

### 4-((tert-Butyldimethylsilyl)oxy)-5-methoxy-2,3-dihydro-1H-inden-1-one (16)

Phenol 15 (0.90 g, 5.1 mmol) was dissolved in DMF (25 mL), and TBSCl (0.71 g, 4.7 mmol) was added, followed by the addition of DIPEA (1.24 mL, 7.08 mmol). The mixture was stirred for 12 h at room temperature, washed with water, and extracted with EtOAc (5×50 mL). The organic layer was dried over sodium sulfate, concentrated and purified by flash chromatography using a prepacked 50 g silica column [solvent A: EtOAc; solvent B: hexane; gradient: 7% A/93% B (1 CV), 7% A/93% B→60% A/40% B (10 CV), 60% A/40% B (2 CV); flow rate: 50 mL/min; monitored at 254 nm and 280 nm] to afford TBS-protected 16 (0.63 g, 2.2 mmol, 91%) as a white solid. ¹H NMR (CDCl₃, 500 MHz) δ 7.30 (1H, d, J=8.5 Hz), 6.83 (1H, d, J=8.5 Hz), 3.81 (3H, s), 2.94 (2H, t, J=6 Hz), 2.56 (2H, t, J=6 Hz), 0.95 (9H, s), 0.12 (6H s). ¹³C NMR (CDCl₃, 150 MHz) δ 205.8, 155.0, 146.3, 141.4, 131.1, 117.4, 111.5, 55.4, 36.4, 25.9, 22.9, 18.6, −4.1.

### 4-((tert-Butyldimethylsilyl)oxy)-5-methoxy-1-(3′,4′,5′-trimethoxyphenyl)-2,3-dihydro-1H-inden-1-ol (27)

5-Bromo-1,2,3-trimethoxybenzene (0.51 g, 2.1 mmol) was dissolved in THF (25 mL) and cooled to −78° C. n-BuLi (1.22 mL, 3.05 mmol) was added dropwise, and the reaction mixture was stirred for 1 h. TBS-protected 16 (0.43 g, 1.53 mmol) was dissolved in THF (10 mL) and added dropwise to the reaction flask, and the mixture was stirred for 12 h while warming to room temperature, at which time it was washed with water, extracted with EtOAc (3×30 mL), dried over sodium sulfate, concentrated, and purified by flash chromatography using a prepacked 50 g silica column [solvent A: EtOAc; solvent B: hexane; gradient: 7% A/93% B (2 CV), 7% A/93% B→60% A/40% B (10 CV), 60% A/40% B (2 CV); flow rate: 50 mL/min; monitored at 254 nm and 280 nm] to afford tertiary alcohol 27 (0.37 g, 0.80 mmol, 37%) as a yellow oil. NMR characterization was performed after the next step.

### tert-Butyl((6-methoxy-3-(3′,4′,5′-trimethoxyphenyl)-1H-inden-7-yl)oxy)dimethylsilane (30)

Acetic acid (15 mL) was added to tertiary alcohol 27 (0.37 g, 0.80 mmol), and the reaction mixture was stirred at room temperature for 12 h, at which time it was washed with water and extracted with EtOAc (3×30 mL). The combined organic layer was dried over sodium sulfate, concentrated, and purified by flash chromatography using a prepacked 25 g silica column [solvent A: EtOAc; solvent B: hexane; gradient: 7% A/93% B (1 CV), 7% A/93% B→60% A/40% B (10 CV), 60% A/40% B (2 CV); flow rate: 25 mL/min; monitored at 254 nm and 280 nm] to afford TBS-protected 30 (0.14 g, 0.32 mmol, 39%) as a clear oil. ¹H NMR (CDCl₃, 600 MHz) δ 6.92 (1H, d, J=6.5 Hz), 6.64 (1H, d, J=7 Hz), 6.59 (2H, s), 6.19 (1H, t, J=2 Hz), 3.69 (9H, s), 3.62 (3H, s), 3.23 (2H, d, J=2 Hz), 0.84 (9H, s), 0.00 (6H, s). ¹³C NMR (CDCl₃, 150 MHz) δ 153.3, 148.8, 144.9, 141.2, 138.2, 137.5, 135.7, 132.1, 128.8, 113.2, 110.3, 104.7, 61.0, 56.2, 55.6, 35.9, 26.1, 18.7, −4.1.

### 6-Methoxy-3-(3′,4′,5′-trimethoxyphenyl)-1H-inden-7-ol (31) (WO2012/068284)

TBS-protected 30 (0.46 g, 1.0 mmol) was dissolved in THF (5 mL), TBAF (5.2 mL, 5.2 mmol) was added, and the reaction mixture was stirred at room temperature for 12 h, at which time the mixture was washed with water, extracted with EtOAc (3×40 mL), dried with sodium sulfate, evaporated under reduced pressure, and purified by flash chromatography using a prepacked 25 g silica column [solvent A: EtOAc; solvent B: hexane; gradient: 12% A/88% B (1 CV), 12% A/88% B→100% A/0% B (10 CV), 100% A/0% B (2 CV); flow rate: 25 mL/min; monitored at 254 nm and 280 nm] to afford indene 31 (0.23 g, 0.70 mmol, 68%) as a yellow oil. ¹H NMR (CDCl₃, 600 MHz) δ 7.08 (1H, d, J=7.8 Hz), 6.87 (1H, d, J=7.8 Hz), 6.80 (2H, s), 6.46 (1H, t, J=2.4 Hz), 5.78 (1H, s), 3.94 (3H, s), 3.91 (9H, s), 3.48 (2H, d, J=2.4 Hz). ¹³C NMR (CDCl₃, 150 MHz) δ 153.3, 144.9, 144.8, 141.8, 139.7, 138.7, 137.6, 131.9, 129.37, 139.35, 111.7, 109.2, 104.8, 61.0, 56.5, 56.2, 34.8. HRMS: Obsvd 351.1203 [M+Na]⁺, calcd for C₁₉H₂₀O₅Na: 351.1208. HPLC: 12.84 min.

### Sodium 6-methoxy-3-(3′,4′,5′-trimethoxyphenyl)-1H-inden-7-yl phosphate (32)

POCl₃ (0.26 mL, 2.8 mmol) was cooled to 0° C. in CH₂Cl₂ (10 mL). Indene 31 (0.23 g, 0.70 mmol) and pyridine (0.20 mL, 2.5 mmol) in CH₂Cl₂ (5 mL) was added to the reaction mixture dropwise, and the reaction mixture was stirred at 0° C. for 1 h. The mixture was then warmed to room temperature over 12 h. The mixture was then evaporated under reduced pressure. DCM (10 mL) was added to the resulting residue, which was again evaporated under reduced pressure. This was repeated two more times. The resulting solid was dissolved in a mixture of THF and water (2:1, 6 mL total) and stirred for 1 h. The solution was then cooled to 0° C. and 0.1 M NaOH was added until a pH of 10 was achieved. The solution was then evaporated under reduced pressure, and the crude product was purified by a C18 30 g reversed phase column [solvent A: acetonitrile; solvent B: water; gradient: 10% A/90% B (1 CV), 10% A/90% B→100% A/0% B (10 CV), 100% A/0% B (2 CV); flow rate: 25 mL/min; monitored at 254 and 280 nm] to afford phosphate salt 32 (0.09 g, 0.20 mmol, 28%) as a light brown solid. ¹H NMR (600 MHz, D₂O) δ 6.98 (1H, d, J=8.4 Hz), 6.81 (1H, d, J=7.8 Hz), 6.67 (2H, s), 6.39 (1H, t, J=1.8 Hz), 3.79 (3H, s), 3.68 (6H, s), 3.65 (3H, s), 3.58 (2H, s). ¹³C NMR (150 MHz, D₂O) δ 152.4, 150.0 (d, J=3 Hz), 142.8, 139.5 (d, J=6.4 Hz), 138.2 (d, J=2.5 Hz), 137.4, 136.0, 132.4, 130.8, 115.0, 111.1, 104.8, 60.9, 56.3, 55.9, 36.3. ³¹P NMR (242 MHz, D₂O) δ 0.60. HRMS: Obsvd 453.0687 [M+H], Calcd for C₁₉H₂₀O₈Na₂P⁺: 453.0686 HPLC: 4.04 min.

### 4,5-Dimethoxy-1-(3′,4′,5′-trimethoxyphenyl)-2,3-dihydro-1H-inden-1-ol (25)

5-Bromo-1,2,3-trimethoxybenzene (1.60 g, 6.47 mmol) was dissolved in THF (50 mL) and cooled to −78° C. n-BuLi (2.7 mL, 6.8 mmol) was added dropwise, and the reaction mixture was stirred for 1 h. Ketone 11 (0.92 g, 4.79 mmol) was dissolved in THF (10 mL) and added dropwise to the reaction flask, and the mixture was stirred for 12 h while warming to room temperature, at which time it was washed with water, extracted with EtOAc (3×30 mL), dried over sodium sulfate, concentrated, and purified by flash chromatography using a prepacked 50 g silica column [solvent A: EtOAc; solvent B: hexane; gradient: 10% A/90% B (1 CV), 10% A/90% B→80% A/20% B (10 CV), 80% A/20% B (1 CV); flow rate: 50 mL/min; monitored at 254 nm and 280 nm] to afford tertiary alcohol 25 (1.221 g, 3.39 mmol, 71%) as an orange oil. NMR data was collected after the subsequent step.

### 6,7-Dimethoxy-3-(3′,4′,5′-trimethoxyphenyl)-1H-indene (28)

Acetic acid (25 mL) was added to tertiary alcohol 25 (1.22 g 3.39 mmol), and the mixture was stirred at room temperature for 12 h. The mixture was washed with water, extracted with EtOAc (3×30 mL), dried over sodium sulfate, concentrated under reduced pressure, and purified by flash chromatography using a prepacked 50 g silica column [solvent A: EtOAc; solvent B: hexane; gradient: 7% A/93% B (1 CV), 7% A/93% B→60% A/40% B (10 CV), 60% A/40% B (2 CV); flow rate: 40 mL/min; monitored at 254 nm and 280 nm] to afford indene 28 (0.56 g, 1.64 mmol, 48%) as a brown solid. ¹H NMR (CDCl₃, 600 MHz) δ 7.28 (1H, d, J=8 Hz), 6.93 (1H, d, J=8.5 Hz), 6.83 (2H, s), 6.44 (1H, s), 3.99 (3H, s), 3.93 (3H, s), 3.91 (9H, s), 3.53 (2H, s). ¹³C NMR (CDCl₃, 150 MHz) δ 153.1, 150.2, 145.2, 144.5, 138.2, 137.4, 136.4, 131.6, 128.6, 115.2, 110.9, 104.5, 60.6, 59.8, 56.0, 55.8, 35.2. HRMS: Obsvd 365.1385 [M+Na]⁺, calcd for C₂₀H₂₂O₅Na: 365.1359. HPLC: 14.96 min.

### 3-(3′-Methoxyphenyl)propanoic acid (10) (Cohen, 1935)

3-methoxycinnamic acid (8) (3.56 g, 19.98 mmol) was dissolved in MeOH (100 mL), and 10% Pd on carbon (0.44 g) was added. The mixture was stirred for 12 h at room temperature under H₂ (balloon). The reaction mixture was then filtered through Celite®, and the Celite® was washed with EtOAc (3×30 mL). The filtrate (EtOAc and MeOH) was evaporated under reduced pressure resulting in carboxylic acid 10 (3.59 g, 19.7 mmol, quantitative). ¹H NMR (CDCl₃, 600 MHz) δ 11.93 (1H, s), 7.27 (1H, t, J=7.5 Hz), 6.88 (2H, m), 6.83 (1H, d, J=8 Hz), 3.79 (3H, s), 2.99 (2H, t, J=7 Hz), 2.73 (2H, t, J=7 Hz). ¹³C NMR (CDCl₃, 150 MHz) δ 179.2, 159.9, 142.0, 129.6, 120.7, 114.2, 111.6, 54.9, 35.5, 30.6.

### 5-Methoxy-2,3-dihydro-1H-inden-1-one (12) (WO01/68654; Ingold et al., 1923)

Eaton's reagent (43 mL, 3 g/mmol of carboxylic acid 10) was added to carboxylic acid 10 (3.95 g, 21.9 mmol), and the reaction mixture was stirred at room temperature for 72 h. It was then poured over ice, neutralized, and extracted with EtOAc (3×50 mL). The organic layer was dried over sodium sulfate, concentrated, and purified by flash chromatography using a prepacked 100 g silica column [solvent A: EtOAc; solvent B: hexane; gradient: 12% A/88% B (1 CV), 12% A/88% B→100% A/0% B (10 CV), 100% A/0% B (10 CV); flow rate: 50 mL/min; monitored at 254 nm and 280 nm] to afford ketone 12 (2.26 g, 13.9 mmol, 64%) as a green solid. ¹H NMR (CDCl₃, 600 MHz) δ 7.49 (1H, d, J=7.5 Hz), 6.72 (2H, m), 3.72 (3H, s), 2.91 (2H, t, J=6 Hz), 2.48 (2H, t, J=5.5 Hz). ¹³C NMR (CDCl₃, 150 MHz) δ 205.0, 165.1, 158.1, 130.2, 125.0, 115.2, 109.6, 55.5, 36.3, 25.7.

### 5-Methoxy-1-(3′,4′,5′-trimethoxyphenyl)-2,3-dihydro-1H-inden-1-ol (26) (WO01/68654)

5-Bromo-1,2,3-trimethoxybenzene (1.95 g, 7.91 mmol) was dissolved in THF (50 mL), and the mixture was cooled to −78° C. n-BuLi (3.3 mL, 8.3 mmol) was added dropwise, and the reaction mixture was stirred for 1 h. Ketone 12 (0.95 g, 5.86 mmol) was dissolved in THF (10 mL) and added dropwise to the reaction flask, and the mixture was stirred for 12 h warming to room temperature, at which time it was washed with water, extracted with EtOAc (3×30 mL), dried over sodium sulfate, concentrated under reduced pressure, and purified by flash chromatography using a prepacked 100 g silica column [solvent A: EtOAc; solvent B: hexane; gradient: 10% A/90% B (1 CV), 10% A/90% B→80% A/20% B (10 CV), 80% A/20% B (2 CV); flow rate: 50 mL/min; monitored at 254 nm and 280 nm] to afford tertiary alcohol 26 (1.45 g, 4.39 mmol, 75%) as a yellow oil. NMR data was collected after the subsequent step.

### 6-Methoxy-3-(3′,4′,5′-trimethoxyphenyl)-1H-indene (29) (WO01/68654)

Acetic acid (25 mL) was added to tertiary alcohol 26 (1.45 g 4.39 mmol) and stirred at room temperature for 12 h. The mixture was washed with water, extracted with EtOAc (3×30 mL), dried over sodium sulfate, concentrated, and purified by flash chromatography using a prepacked 100 g silica column [solvent A: EtOAc; solvent B: hexane; gradient: 7% A/93% B (1 CV), 7% A/93% B→60% A/40% B (10 CV), 60% A/40% B (1 CV); flow rate: 50 mL/min; monitored at 254 nm and 280 nm] to afford indene 29 (1.30 g, 4.16 mmol, 95%) as a red-yellow oil. ¹H NMR (CDCl₃, 600 MHz) δ 7.47 (1H, d, J=8.5 Hz), 7.08 (1H, d, J=2 Hz), 6.86 (1H, dd, J=10.5, 2 Hz), 6.81 (2H, s), 6.38 (1H, t, J=2 Hz), 3.90 (3H, s), 3.87 (6H, s), 3.79 (3H, s), 3.40 (2H, d, J=1 Hz). ¹³C NMR (CDCl₃, 150 MHz) δ 150.1, 153.3, 146.7, 144.6, 137.6, 136.8, 132.0, 128.4, 120.5, 111.8, 110.6, 104.7, 60.8, 56.1, 55.4, 38.0. HRMS: Obsvd 335.1281 [M+Na]⁺, calcd for C₁₉H₂₀O₄Na: 335.1254. HPLC: 15.95 min.

### REFERENCES

The following publications are hereby incorporated by reference.
R. J. Ludford, J. Natl. Cancer Inst., 1945, 6, 89-101.
P. B. Schiff and S. B. Horwitz, Proc. Natl. Acad. Sci., 1980, 77, 1561-1565.
P. B. Schiff and S. B. Horwitz, Biochemistry (Mosc.), 1981, 20, 3247-3252.
G. R. Pettit, S. B. Singh, E. Hamel, C. M. Lin, D. S. Alberts and D. Garcia-Kendal, Experientia, 1989, 45, 209-211.
M. Gorman, N. Neuss, G. H. Svoboda, A. J. Barnes and N. J. Cone, J. Am. Pharm. Assoc., 1959, 48, 256-257.
R. J. Owellen, A. H. Owens and D. W. Donigian, Biochem. Biophys. Res. Commun., 1972, 47, 685-691.
WO2012068284 (A2), 2012.
WO0168654 (A2), 2001.
M. Sriram, J. J. Hall, N. C. Grohmann, T. E. Strecker, T. Wootton, A. Franken, M. L. Trawick and K. G. Pinney, Bioorg. Med. Chem., 2008, 16, 8161-8171.
R. P. Tanpure, C. S. George, T. E. Strecker, L. Devkota, J. K. Tidmore, C.-M. Lin, C. A. Herdman, M. T. MacDonough, M. Sriram, D. J. Chaplin, M. L. Trawick and K. G. Pinney, Bioorg. Med. Chem., 2013, 21, 8019-8032.
C. A. Herdman, L. Devkota, C.-M. Lin, H. Niu, T. E. Strecker, R. Lopez, L. Liu, C. S. George, R. P. Tanpure, E. Hamel, D. J. Chaplin, R. P. Mason, M. L. Trawick and K. G. Pinney, Bioorg. Med. Chem., 2015, 23, 7497-7520.
R. P. Tanpure, C. S. George, M. Sriram, T. E. Strecker, J. K. Tidmore, E. Hamel, A. K. Charlton-Sevcik, D. J. Chaplin, M. L. Trawick and K. G. Pinney, Med Chem Comm, 2012, 3, 720-724.
L. Devkota, C.-M. Lin, T. E. Strecker, Y. Wang, J. K. Tidmore, Z. Chen, R. Guddneppanavar, C. J. Jelinek, R. Lopez, L. Liu, E. Hamel, R. P. Mason, D. J. Chaplin, M. L. Trawick and K. G. Pinney, Bioorg. Med. Chem., 2016, 24, 938-956.
M. B. Hadimani, M. T. MacDonough, A. Ghatak, T. E. Strecker, R. Lopez, M. Sriram, B. L. Nguyen, J. J. Hall, R. J. Kessler, A. R. Shirali, L. Liu, C. M. Garner, G. R. Pettit, E. Hamel, D. J. Chaplin, R. P. Mason, M. L. Trawick and K. G. Pinney, J. Nat. Prod., 2013, 76, 1668-1678.
B. L. Flynn, G. S. Gill, D. W. Grobelny, J. H. Chaplin, D. Paul, A. F. Leske, T. C. Lavranos, D. K. Chalmers, S. A. Charman, E. Kostewicz, D. M. Shackleford, J. Morizzi, E. Hamel, M. K. Jung and G. Kremmidiotis, J. Med. Chem., 2011, 54, 6014-6027.
G. Kremmidiotis, A. F. Leske, T. C. Lavranos, D. Beaumont, J. Gasic, A. Hall, M. O'Callaghan, C. A. Matthews and B. Flynn, Mol. Cancer Ther., 2010, 9, 1562-1573.
J. M. Lambert, Br. J. Clin. Pharmacol., 2013, 76, 248-262.
A. E. Prota, K. Bargsten, J. F. Diaz, M. Marsh, C. Cuevas, M. Liniger, C. Neuhaus, J. M. Andreu, K.-H. Altmann and M. O. Steinmetz, Proc. Natl. Acad. Sci., 2014, 111, 13817-13821.
D. Ma, C. E. Hopf, A. D. Malewicz, G. P. Donovan, P. D. Senter, W. F. Goeckeler, P. J. Maddon and W. C. Olson, Clin. Cancer Res., 2006, 12, 2591-2596.
M. P. Hay, K. O. Hicks and J. Wang, in Tumor Microenvironment and Cellular Stress, eds. C. Koumenis, E. Hammond and A. Giaccia, Springer New York, 2014, pp. 111-145.
W. R. Wilson and M. P. Hay, Nat. Rev. Cancer, 2011, 11, 393-410.
M. J. McKeage, M. B. Jameson, R. K. Ramanathan, J. Rajendran, Y. Gu, W. R. Wilson, T. J. Melink and N. S. Tchekmedyian, BMC Cancer, 2012, 12, 1-10.
M. R. Abbattista, S. M. F. Jamieson, Y. Gu, J. E. Nickel, S. M. Pullen, A. V. Patterson, W. R. Wilson and C. P. Guise, Cancer Biol. Ther., 2015, 16, 610-622.
S. P. Chawla, L. D. Cranmer, B. A. V. Tine, D. R. Reed, S. H. Okuno, J. E. Butrynski, D. R. Adkins, A. E. Hendifar, S. Kroll and K. N. Ganjoo, J. Clin. Oncol., 2014, 1-11.
Q. Liu, J. D. Sun, J. Wang, D. Ahluwalia, A. F. Baker, L. D. Cranmer, D. Ferraro, Y. Wang, J.-X. Duan, W. S. Ammons, J. G. Curd, M. D. Matteucci and C. P. Hart, Cancer Chemother. Pharmacol., 2012, 69, 1487-1498.
F. Meng, J. W. Evans, D. Bhupathi, M. Banica, L. Lan, G. Lorente, J.-X. Duan, X. Cai, A. M. Mowday, C. P. Guise, A. Maroz, R. F. Anderson, A. V. Patterson, G. C. Stachelek, P. M. Glazer, M. D. Matteucci and C. P. Hart, Mol. Cancer Ther., 2012, 11, 740-751.
P. Thomson, M. A. Naylor, S. A. Everett, M. R. L. Stratford, G. Lewis, S. Hill, K. B. Patel, P. Wardman and P. D. Davis, Mol. Cancer Ther., 2006, 5, 2886-2894.
D. W. Siemann, M. C. Bibby, G. G. Dark, A. P. Dicker, F. A. Eskens, M. R. Horsman, D. Marmé and P. M. LoRusso, Clin. Cancer Res., 2005, 11, 416-420.
M. R. Horsman, A. B. Bohn and M. Busk, Exp Oncol, 2010, 32, 143-148.
P. E. Thorpe, Clin. Cancer Res., 2004, 10, 415-427.
J. Denekamp, Cancer Metastasis Rev., 1990, 9, 267-282.
D. W. Siemann, D. J. Chaplin and M. R. Horsman, Cancer, 2004, 100, 2491-2499.
M. H. Fens, G. Storm and R. M. Schiffelers, Expert Opin. Investig. Drugs, 2010, 19, 1321-1338.
J. Denekamp, Br. J. Cancer, 1982, 45, 136-139.
G. M. Tozer, C. Kanthou and B. C. Baguley, Nat. Rev. Cancer, 2005, 5, 423-435.
D. W. Siemann, Cancer Treat. Rev., 2011, 37, 63-74.
N. S. Vasudev and A. R. Reynolds, Angiogenesis, 2014, 17, 471-494.
R. K. Jain, Cancer Cell, 2014, 26, 605-622.
J. Folkman, Nat. Rev. Drug Discov., 2007, 6, 273-286.
M. J. Pilat and P. M. LoRusso, J. Cell. Biochem., 2006, 99, 1021-1039.
G. R. Pettit and M. R. Rhodes, Anticancer. Drug Des., 1998, 13, 183-191.
V. K. Kretzschmann and R. Fürst, Phytochem. Rev., 2014, 13, 191-206.
Y.-T. Ji, Y.-N. Liu and Z.-P. Liu, Curr. Med. Chem., 2015, 22, 1348-1360.
P. Nathan, M. Zweifel, A. R. Padhani, D.-M. Koh, M. Ng, D. J. Collins, A. Harris, C. Carden, J. Smythe, N. Fisher, N. J. Taylor, J. J. Stirling, S.-P. Lu, M. O. Leach, G. J. S. Rustin and I. Judson, Clin. Cancer Res. Off J. Am. Assoc. Cancer Res., 2012, 18, 3428-3439.
B. C. Baguley and M. J. McKeage, Clin. Investig., 2012, 2, 985-993.
G. R. Pettit and J. W. Lippert, Anticancer. Drug Des., 2000, 15, 203-216.
D. Rischin, D. C. Bibby, G. Chong, G. Kremmidiotis, A. F. Leske, C. A. Matthews, S. Wong, M. Rosen and J. Desai, Clin. Cancer Res., 2011, 17, 5152-5160.
K. Hori, S. Saito, Y. Nihei, M. Suzuki and Y. Sato, Jpn. J. Cancer Res., 1999, 90, 1026-1038.
K. Hori and S. Saito, Br. J. Cancer, 2003, 89, 1334-1344.
J.-Y. Blay, Z. Pipai, A. W. Tolcher, A. Italiano, D. Cupissol, A. López-Pousa, S. P. Chawla, E. Bompas, N. Babovic, N. Penel, N. Isambert, A. P. Staddon, E. Saada-Bouzid, A. Santoro, F. A. Franke, P. Cohen, S. Le-Guennec and G. D. Demetri, Lancet Oncol., 2015, 16, 531-540.
P. D. Davis, G. J. Dougherty, D. C. Blakey, S. M. Galbraith, G. M. Tozer, A. L. Holder, M. A. Naylor, J. Nolan, M. R. L. Stratford, D. J. Chaplin and S. A. Hill, Cancer Res., 2002, 62, 7247-7253.
R. M. Lee and D. A. Gewirtz, Drug Dev. Res., 2008, 69, 352-358.
J. Ludford, J. Natl. Cancer Inst., 1945, 6, 89-101.
R. P. Mason, D. Zhao, L. Liu, M. L. Trawick and K. G. Pinney, Integr. Biol., 2011, 3, 375-387.
G. G. Dark, S. A. Hill, V. E. Prise, G. M. Tozer, G. R. Pettit and D. J. Chaplin, Cancer Res., 1997, 57, 1829-1834.
I. G. Kirwan, P. M. Loadman, D. J. Swaine, D. A. Anthoney, G. R. Pettit, J. W. Lippert, S. D. Shnyder, P. A. Cooper and M. C. Bibby, Clin. Cancer Res., 2004, 10, 1446-1453.
K. Grosios, S. E. Holwell, A. T. McGown, G. R. Pettit and M. C. Bibby, Br. J. Cancer, 1999, 81, 1318-1327.
S. Iyer, D. J. Chaplin, D. S. Rosenthal, A. H. Boulares, L.-Y. Li and M. E. Smulson, Cancer Res., 1998, 58, 4510-4514.
M. M. Cooney, J. Ortiz, R. M. Bukowski and S. C. Remick, Curr. Oncol. Rep., 2005, 7, 90-95.
A. Dowlati, K. Robertson, M. Cooney, W. P. Petros, M. Stratford, J. Jesberger, N. Rafie, B. Overmoyer, V. Makkar, B. Stambler, A. Taylor, J. Waas, J. S. Lewin, K. R. McCrae and S. C. Remick, Cancer Res., 2002, 62, 3408-3416.
G. J. S. Rustin, S. M. Galbraith, H. Anderson, M. Stratford, L. K. Folkes, L. Sena, L. Gumbrell and P. M. Price, J. Clin. Oncol., 2003, 21, 2815-2822.
P. Nathan, M. Zweifel, A. R. Padhani, D.-M. Koh, M. Ng, D. J. Collins, A. Harris, C. Carden, J. Smythe, N. Fisher, N. J. Taylor, J. J. Stirling, S.-P. Lu, M. O. Leach, G. J. S. Rustin and I. Judson, Clin. Cancer Res., 2012, 18, 3428-3439.
K. G. Pinney, M. P. Mejia, V. M. Villalobos, B. E. Rosenquist, G. R. Pettit, P. Verdier-Pinard and E. Hamel, Bioorg. Med. Chem., 2000, 8, 2417-2425.
R. Siles, J. F. Ackley, M. B. Hadimani, J. J. Hall, B. E. Mugabe, R. Guddneppanavar, K. A. Monk, J.-C. Chapuis, G. R. Pettit, D. J. Chaplin, K. Edvardsen, M. L. Trawick, C. M. Garner and K. G. Pinney, J. Nat. Prod., 2008, 71, 313-320.
R. P. Tanpure, A. R. Harkrider, T. E. Strecker, E. Hamel, M. L. Trawick and K. G. Pinney, Bioorg. Med. Chem., 2009, 17, 6993-7001.
R. P. Tanpure, B. L. Nguyen, T. E. Strecker, S. Aguirre, S. Sharma, D. J. Chaplin, B. G. Siim, E. Hamel, J. W. Lippert, G. R. Pettit, M. L. Trawick and K. G. Pinney, J. Nat. Prod., 2011, 74, 1568-1574.
K. G. Pinney, A. D. Bounds, K. M. Dingeman, V. P. Mocharla, G. R. Pettit, R. Bai and E. Hamel, Bioorg. Med. Chem. Lett., 1999, 9, 1081-1086.
M. T. MacDonough, T. E. Strecker, E. Hamel, J. J. Hall, D. J. Chaplin, M. L. Trawick and K. G. Pinney, Bioorg. Med. Chem., 2013, 21, 6831-6843.
C. A. Herdman, L. Devkota, C.-M. Lin, H. Niu, T. E. Strecker, R. Lopez, L. Liu, C. S. George, R. P. Tanpure, E. Hamel, D. J. Chaplin, R. P. Mason, M. L. Trawick and K. G. Pinney, Bioorg. Med. Chem.
P. E. Eaton, G. R. Carlson and J. T. Lee, J. Org. Chem., 1973, 38, 4071-4073.
G. J. Kemperman, T. A. Roeters and P. W. Hilberink, Eur. J. Org. Chem., 2003, 2003, 1681-1686.
U. Neuenschwander and I. Hermans, J. Org. Chem., 2011, 76, 10236-10240.
J. Cossy, S. Arseniyadis and C. Meyer, Metathesis in Natural Product Synthesis: Strategies, Substrates and Catalysts, John Wiley & Sons, 2011.
G. R. Pettit, M. P. Grealish, D. L. Herald, M. R. Boyd, E. Hamel and R. K. Pettit, J. Med. Chem., 2000, 43, 2731-2737.
M. A. Paley and J. A. Prescher, Med Chem Comm, 2014, 5, 255-267.
X. Ma, H. Hui, W. Shang, X. Jia, X. Yang and J. Tian, Curr. Drug Targets, 2015, 16, 542-548.
D. Zhao, E. Richer, P. P. Antich and R. P. Mason, FASEB J., 2008, 22, 2445-2451.
M. K. Alhasan, L. Liu, M. A. Lewis, J. Magnusson and R. P. Mason, PLoS ONE, 2012, 7, e46106.
L. Liu, H. Beck, X. Wang, H.-P. Hsieh, R. P. Mason and X. Liu, PloS One, 2012, 7, e43314.
H. Zhou, R. R. Hallac, R. Lopez, R. Denney, M. T. MacDonough, L. Li, L. Liu, E. E. Graves, M. L. Trawick, K. G. Pinney and R. P. Mason, Am. J. Nucl. Med. Mol. Imaging, 2015, 5, 143-153.
D. M. Gapinski, B. E. Mallett, L. L. Froelich and W. T. Jackson, J. Med. Chem., 1990, 33, 2807-2813.
R. A. Ross, N. Henry, S. N. Nabi, S. N. Nabi, N. K. Das, I. R. Beattie, P. A. Cocking, I. L. Finar, K. J. Saunders, A. Goldup, A. B. Morrison, G. W. Smith, A. R. Blake, K. N. Bascombe, M. Cowperthwaite, R. Shaw, C. B. Barlow, R. D. Guthrie, D. Murphy, V. Askam, D. Bailey, D. Jaques, J. D. Donaldson, J. D. O'Donogue, R. Oteng, J. Powell, B. L. Shaw, T. van Es, P. S. Bramwell, A. O. Fitton, E. E. Glover, G. H. Morris, E. N. Morgan, P. J. Palmer, L. Kruszynska, W. R. N. Williamson, J. A. McCleverty, A. Davison, G. Wilkinson, L. T. Allan, G. A. Swan, J. Lewis, F. Mabbs, H. D. Law, H. Goldwhite, R. A. Heacock and O. Hutzinger, J. Chem. Soc. Resumed, 1965, 3854-3904.
R. A. Bauer, T. A. Wenderski and D. S. Tan, Nat. Chem. Biol., 2013, 9, 21-29.
A. Mandal, S. Bhattacharya, S. R. Raychaudhuri and A. Chatterjee, J. Chem. Res. Synop., 1988, 366-367.
V. B. Kammath, T. S\check{o}lomek, B. P. Ngoy, D. Heger, P. Klán, M. Rubina and R. S. Givens, J. Org. Chem., 2013, 78, 1718-1729.
W. H. J. Perkin and R. Robinson, J. Chem. Soc. Trans., 1914, 105, 2376-92.
J. D. Williams, A. R. Khan, S. C. Cardinale, M. M. Butler, T. L. Bowlin and N. P. Peet, Bioorg. Med. Chem., 2014, 22, 419-434.
J. Perkin, William H. and R. Robinson, J. Chem. Soc. Trans., 1914, 105, 2376-2392.
M. L. Pati, C. Abate, M. Contino, S. Ferorelli, R. Luisi, L. Carroccia, M. Niso and F. Berardi, Eur. J. Med. Chem., 2015, 89, 691-700.
N. Fujii, H. Irie and H. Yajima, J. Chem. Soc. [Perkin 1], 1977, 2288-2289.
J. P. Day, B. Lindsay, T. Riddell, Z. Jiang, R. W. Allcock, A. Abraham, S. Sookup, F. Christian, J. Bogum, E. K. Martin, R. L. Rae, D. Anthony, G. M. Rosair, D. M. Houslay, E. Huston, G. S. Baillie, E. Klussmann, M. D. Houslay and D. R. Adams, J. Med. Chem., 2011, 54, 3331-3347.
A. Cohen, J. Chem. Soc., 1935, 429-436.
C. K. Ingold and H. A. Piggott, J. Chem. Soc. Trans., 1923, 123, 1469-1509.
A. Monks, D. Scudiero, P. Skehan, R. Shoemaker, K. Paull, D. Vistica, C. Hose, J. Langley, P. Cronise, A. Vaigro-Wolff, M. Gray-Goodrich, H. Campbell, J. Mayo and M. Boyd, J. Natl. Cancer Inst., 1991, 83, 757-766.
V. Vichai and K. Kirtikara, Nat. Protoc., 2006, 1, 1112-1116.
E. Hamel and C. M. Lin, Biochim. Biophys. Acta BBA—Gen. Subj., 1981, 675, 226-231.
E. Hamel, Cell Biochem. Biophys., 2003, 38, 1-21.
Herdman C A, Strecker T E, Tanpure R P, Chen Z, Winters A, Gerberich J, Liu L, Hamel E, Mason R P, Chaplin D J, Trawick M L, Pinney K G, Synthesis and Biological Evaluation of Benzocyclooctene-based and Indene-based Anticancer Agents that Function as Inhibitors of Tubulin Polymerization, Med. Chem. Commun., 2016, 7, 2418-2427.

What is claimed is:

1. A method of treating a vascular proliferative disorder in a subject, comprising:
administering a pharmaceutical composition to the subject, wherein the pharmaceutical composition comprises a therapeutically effective amount of a benzocyclooctene compound demonstrating inhibition of tubulin polymerization and having a structure of: 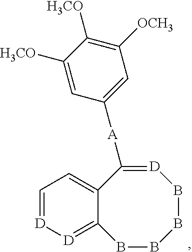
wherein A is a bond, C═O, C═NH, or C═NR₁, where R₁ is any alkyl, aryl, or alkaryl substituent,
B is CH₂, NH, O, S, CH—OH, or CH—OR₂, wherein R₂ is CH₃, or PO₃⁻, or C—Z, wherein Z is halogen, and
D is CH, N, C—OH, or C—OR₃, wherein R₃ is CH₃, any alkyl substituent, any alkoxy substituent, any alkyl or alkoxy substituent including heteroatoms, any carbonyl substituent, any ester substituent, or PO₃⁻ or C—Z, wherein Z is halogen, and
wherein the pharmaceutical composition further comprises a pharmaceutically acceptable excipient, adjuvant, carrier, buffer, stabilizer, or mixture thereof.

2. The method of claim 1, wherein the benzocyclooctene compound has a structure of:

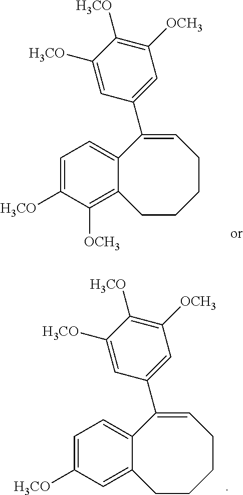 [SMILES: COc1cc(/C2=C/CCCCc3c2ccc(OC)c3OC)cc(OC)c1OC ; COc1ccc2c(c1)CCCC/C=C\2c1cc(OC)c(OC)c(OC)c1]

3. The method of claim 1, wherein the benzocyclooctene compound has a structure of:

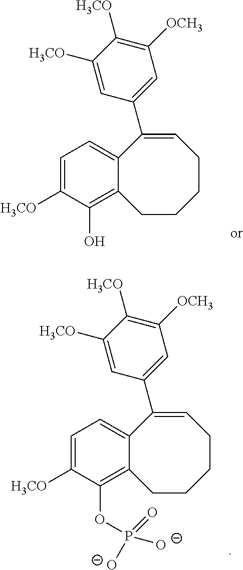 [SMILES: COc1ccc2c(c1O)CCCC/C=C\2c1cc(OC)c(OC)c(OC)c1 ; COc1cc(/C2=C/CCCCc3c2ccc(OC)c3OP(=O)([O-])[O-])cc(OC)c1OC]

4. The method of claim 1, further comprising administering one or more additional therapies to the subject in combination with the pharmaceutical composition.

5. The method of claim 1, wherein the vascular proliferative disorder is cancer.

6. A method of inhibiting tubulin polymerization in a subject, comprising:
administering a pharmaceutical composition to the subject, wherein the pharmaceutical composition comprises a therapeutically effective amount of a benzocyclooctene compound demonstrating inhibition of tubulin polymerization and having a structure of: 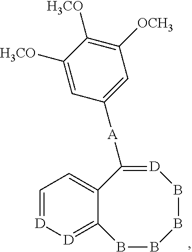
wherein A is a bond, C═O, C═NH, or C═NR₁, where R₁ is any alkyl, aryl, or alkaryl substituent,
B is CH₂, NH, O, S, CH—OH, or CH—OR₂, wherein R₂ is CH₃, or PO₃⁻, or C—Z, wherein Z is halogen, and
D is CH, N, C—OH, or C—OR₃, wherein R₃ is CH₃, any alkyl substituent, any alkoxy substituent, any alkyl or alkoxy substituent including heteroatoms, any carbonyl substituent, any ester substituent, or PO₃⁻ or C—Z, wherein Z is halogen, and
wherein the pharmaceutical composition further comprises a pharmaceutically acceptable excipient, adjuvant, carrier, buffer, stabilizer, or mixture thereof.

7. The method of claim 6, wherein the benzocyclooctene compound has a structure of:

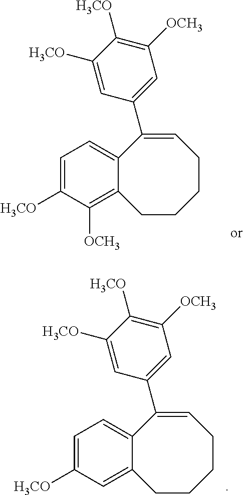 [SMILES: COc1cc(/C2=C/CCCCc3c2ccc(OC)c3OC)cc(OC)c1OC ; COc1ccc2c(c1)CCCC/C=C\2c1cc(OC)c(OC)c(OC)c1]

8. The method of claim 6, wherein the benzocyclooctene compound has a structure of:

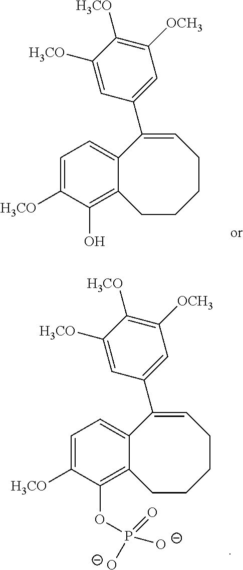 [SMILES: COc1ccc2c(c1O)CCCC/C=C\2c1cc(OC)c(OC)c(OC)c1 ; COc1cc(/C2=C/CCCCc3c2ccc(OC)c3OP(=O)([O-])[O-])cc(OC)c1OC]

9. The method of claim 6, further comprising administering one or more additional therapies to the subject in combination with the pharmaceutical composition.
